(19) United States
(12) Patent Application Publication
(10) Pub. No.: US 2016/0296443 A1
(43) Pub. Date: Oct. 13, 2016

(54) SKIN CARE COMPOSITION HAVING SKIN DAMAGE PREVENTION AND DAMAGE REPAIRING PROPERTIES

(71) Applicant: CGTN C.V., Willemstad (CW)

(72) Inventors: Sreedhar R. Nalabolu, Irving, TX (US); Joy Jamieson, McKinney, TX (US)

(73) Assignee: CGTN C.V., Willemstad (CW)

(21) Appl. No.: 15/093,475

(22) Filed: Apr. 7, 2016

Related U.S. Application Data
(60) Provisional application No. 62/144,809, filed on Apr. 8, 2015.

(51) Int. Cl. A61K 8/46; A61K 8/49; A61K 8/97; A61Q 19/00; A61Q 19/08

(52) U.S. Cl. CPC A61K 8/46; A61Q 19/007; A61Q 19/004; A61Q 19/08; A61K 8/97; A61K 8/498; A61K 8/4973

(57) ABSTRACT

A topical composition comprising (a) an extract of bidens pilosa; (b) isoprenylcysteine; and (c) a dermatologically acceptable carrier is disclosed. The isoprenylcysteine has a concentration from about 0.1 w/v % to about 10 w/v %. A method of using the topical composition to treat an area of skin to reduce DNA fragmentation is also disclosed.

### PRIOR RELATED APPLICATIONS

This application claims the benefit of U.S. Provisional Application No. 62/144,809 entitled “Skin Care Composition Having Skin Damage Prevention and Damage Repairing Properties,” filed on Apr. 8, 2015.

### FEDERALLY SPONSORED RESEARCH STATEMENT

Not Applicable (N/A)

### REFERENCE TO MICROFICHE APPENDIX

N/A

### FIELD OF INVENTION

This disclosure relates in general to topical skin care compositions and methods for application to human skin and, in particular, but not by way of limitation, to skin care composition comprising plant extracts for improving the condition and appearance of skin.

### BACKGROUND OF THE DISCLOSURE

Skin is subject to deterioration through dermatological disorders, environmental abuse (wind, air conditioning, central heating) or through the normal aging process (chrono-aging) which may be accelerated by exposure of skin to sun (photo-aging). In recent years the demand for cosmetic compositions and cosmetic methods for improving the appearance and condition of skin has grown enormously.

Consumers are increasingly seeking “anti-aging” cosmetic products which treat or delay the visible signs of chrono-aging and photo-aging skin such as wrinkles, lines, sagging, hyperpigmentation, and age spots.

Consumers also frequently seek other benefits from cosmetic products in addition to anti-aging. The concept of “sensitive skin” has also raised the consumer demand for cosmetic products which improve the appearance and condition of sensitive, dry, rough and/or flaky skin and to soothe red, irritated and/or itchy skin. Consumers also desire cosmetic products which treat spots, pimples, blemishes etc.

### SUMMARY OF THE INVENTION

In embodiments of the present invention, concentrations are given in weight percent (%) of the total composition, unless specifically stated otherwise (e.g., weight/volume percent (w/v %)). All ratios are weight ratios, unless specifically stated otherwise. Any and all ranges include any range or value within the provided range, unless specifically stated otherwise. The number of significant digits conveys neither a limitation on the indicated amounts nor on the accuracy of the measurements.

In an embodiment, a topical composition comprises (a) an extract of bidens pilosa; (b) isoprenylcysteine; and (c) a dermatologically acceptable carrier. In certain aspects, the isoprenylcysteine has a concentration from about 0.1 w/v % to about 10 w/v % (and any range or value there between). In certain aspects, the isoprenylcysteine has a concentration of about 3 w/v %.

In certain aspects, the topical composition further comprises mauritia flexuosa fruit oil.

In certain aspects, the topical composition further comprises elaeis guineessis oil.

In certain aspects, the topical composition further comprises gossypium herbaceum seed oil.

In certain aspects, the topical composition further comprises linum usitatissimum seed oil.

In certain aspects, the topical composition further comprises tocopherol.

In certain aspects, the topical composition further comprises centella asiatica leaf cell extract.

In certain aspects, the topical composition further comprises one or more of aloe barbadensis leaf juice, natural albumen, and tocopherol.

In certain aspects, the topical composition further comprises one or more of jojoba esters, camellia sinensis leaf extract, ubiquinone, bisabolol, and palmitoyl dipeptide-5 diaminobutyroyl hydroxythreonine.

In certain aspects, the topical composition further comprises pancratium maritimum extract.

In certain aspects, the topical composition further comprises tetrahexyldecyl ascorbate.

In certain aspects, the topical composition further comprises camellia sinensis (green tea) leaf extract.

In certain aspects the isoprenylcysteine is a compound of formula:

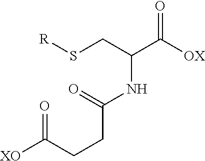

and any variations thereof, including any and all isomers, enantiomers, and potential conversions to salts, esters, polymorphs, hydrates and/or solvates of the isoprenylcysteine, wherein R is an aliphatic group and X is hydrogen, sodium, potassium.

In certain aspects the isoprenylcysteine is N-[1-Carboxy-2-((3,7,11,15,19-pentamethyl-eicos-2-enyl)sulfanyl)-ethyl]succinamic acid or salt of formula:

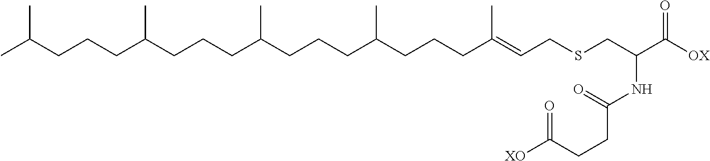

and any variations thereof, including all isomers, enantiomers and potential conversions to salts, esters, polymorphs, hydrates and/or solvates of the N-[1-Carboxy-2-((3,7,11,15,19-pentamethyl-eicos-2-enyl)sulfanyl)-ethyl]succinamic acid or salt.

In certain aspects the isoprenylcysteine is N-[1-Carboxy-2-((3,7,11,15-tetramethyl-hexadec-2-enyl)sulfanyl)-ethyl]succinamic acid or salt of formula:

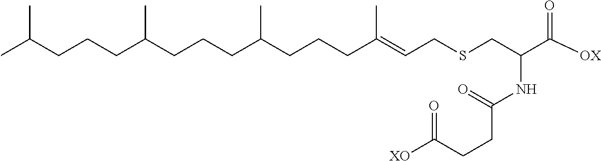

and any variations thereof, including all isomers, enantiomers and potential conversions to salts, esters, polymorphs, hydrates and/or solvates of the N-[1-Carboxy-2-((3,7,11,15-tetramethyl-hexadec-2-enyl)sulfanyl)-ethyl]succinamic acid or salt.

In certain aspects, the isoprenylcysteine is N-[1-Carboxy-2-((3,7,11-trimethyl-dodec-2-enyl)sulfanyl)-ethyl]succinamic acid or salt of formula:

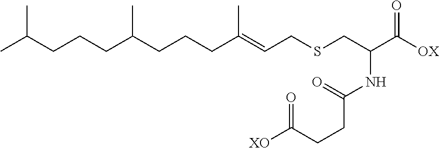

and any variations thereof, including all isomers, enantiomers and potential conversions to salts, esters, polymorphs, hydrates and/or solvates of the N-[1-Carboxy-2-((3,7,11-trimethyl-dodec-2-enyl)sulfanyl)-ethyl]succinamic acid or salt.

In certain aspects, the isoprenylcysteine is Disodium Trimethyldodecenyl Succinoyl Cysteine, Disodium Tetramethylhexadecenyl Succinoyl Cysteine and/or Disodium Pentamethyleicosenyl Succinoyl Cysteine.

In an embodiment, a method for treating an area of skin to reduce DNA fragmentation induced by ultra-violet radiation includes topically applying a composition to the area of skin, the composition comprising: an extract of bidens pilosa, isoprenylcysteine and a dermatologically acceptable carrier. In certain aspects, the isoprenylcysteine has a concentration from about 0.1 w/v % to about 10 w/v % (and any range or value there between). In certain aspects, the isoprenylcysteine has a concentration of about 3 w/v %.

In certain aspects, the composition further comprises centella asiatica leaf cell extract.

In certain aspects, the composition further comprises one or more of aloe barbadensis leaf juice, natural albumen, octyldodecyl olivate, hydrolyzed quinoa, and tocopherol.

In certain aspects, the composition further comprises one or more of oryza sativa bran oil, Montanov™ L, Montanov™ 68, stearic acid, glycerin, glyceryl stearate, ricinus communis seed oil, cetyl alcohol, olus oil, chondrus crispus powder, sodium isostearoyl lactylate, sodium salt of pyroglutamic acid (sodium PCA), polysorbate-20, Dermsoft™ LP, natural cucumber, and esseachem.

In certain aspects, the composition further comprises one or more jojoba esters, camellia sinensis leaf extract, hydrogenated lecithin, ubiquinone, bisabolol, tetrapeptide-30, and palmitoyl dipeptide-5 diaminobutyroyl hydroxythreonine.

In certain aspects, the composition further comprises aloe vera juice.

In certain aspects, the composition further comprises mauritia flexuosa fruit oil.

In certain aspects, the method further includes cleansing the area of the skin prior to applying the composition.

In certain aspects, the method further includes applying the composition once daily in the morning.

In certain aspects, the method further includes applying the composition once daily in the evening.

In certain aspects, the method further includes applying the composition once in the morning and once in the evening.

In certain aspects, the method further includes applying the composition as needed for moisturizing the area of skin.

In certain aspects, the method is further used to treat the area of skin by reducing appearance of fine lines and wrinkles, discoloration, uneven texture, enlarged pores, aging or loose skin.

In an embodiment, a topical composition comprises (a) extract of bidens pilosa; (b) mauritia flexuosa fruit oil; (c) jojoba esters; (d) isoprenylcysteine; (e) centella asiatica leaf cell extract; and (f) tetrahexyldecyl ascorbate. In certain aspects, the isoprenylcysteine has a concentration from about 0.1 w/v % to about 10 w/v % (and any range or value there between). In certain aspects, the isoprenylcysteine has a concentration of about 3 w/v %.

In an embodiment, a topical composition comprises (a) an extract of bidens pilosa; (b) mauritia flexuosa fruit oil; (c) centella asiatica leaf cell extract; (d) isoprenylcysteine; and (e) pancratium maritimum extract. In certain aspects, the isoprenylcysteine has a concentration from about 0.1 w/v % to about 10 w/v % (and any range or value there between). In certain aspects, the isoprenylcysteine has a concentration of about 3 w/v %.

Other aspects, features, and advantages will become apparent from the following detailed description when taken in conjunction with the accompanying drawings, which are a part of this disclosure and which illustrate, by way of example, principles of the inventions disclosed.

### BRIEF DESCRIPTION OF THE DRAWINGS

For a further understanding of the nature and objects of the present invention, reference should be made to the following detailed disclosure, taken in conjunction with the accompanying drawings, in which like parts are given like reference numerals, and wherein:

FIG. 1 is a table according to one illustrative embodiment of a composition or complex that may be used in part, in total or as part of a composition described herein;

FIGS. 2A-2C are a table according to one illustrative embodiment of a composition that may be used either in the morning or night, but preferably at night;

FIGS. 3A-3C are a table according to one illustrative embodiment of a composition that may be used either in the morning or night, but preferably at night;

FIGS. 4A-4D are a table according to one illustrative embodiment of a composition that may be used either in the morning or night, but preferably at night;

FIGS. 5A-5D are a table according to one illustrative embodiment of a composition that may be used either in the morning or night, but preferably in the morning;

FIGS. 6A-6D are a table according to one illustrative embodiment of a composition that may be used either in the morning or night, but preferably in the morning; and

FIGS. 7A-7E are a table according to one illustrative embodiment of a composition that may be used either in the morning or night, but preferably in the morning.

### DETAILED DESCRIPTION OF EMBODIMENTS OF THE INVENTION

The following detailed description of various embodiments of the present invention references the accompanying drawings, which illustrate specific embodiments in which the invention can be practiced. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains. Therefore, the scope of the present invention is defined only by the appended claims, along with the full scope of equivalents to which such claims are entitled.

The compositions, formulas, compounds as described herein may be used in a variety of personal care and skin care products. The compositions may be in a variety of forms, including, but not limited to, an emulsion, lotion, milk, liquid, solid, cream, gel, mousse, ointment, paste, serum, stick, spray, tonic, aerosol, foam, etc.

Definitions

“Vitamin A” is a generic description for all C20-β-ionone derivatives, which qualitatively exhibits the biological activity of transretinol. Vitamin A (retinol) and its derivatives, whether they are natural or synthetic, are referred to as retinoids (RET) and may have significant impacts on vertebrates organisms, including embryogenesis, reproduction, vision, regulation of inflammation, growth and cellular, normal, and neoplastic differentiation.

From a biological stand point, RET takes its effect by binding to specific nuclear receptors, known as RARs (Retinoic Acid Receptors) and RXRs (Retinoids Receptors), which belong to the family of transcriptional regulators induced by binders. RET function by crossing the cellular membrane into the cytoplasm, binding to the cytosolic proteins denominated CRBP or CRABP (Cytosolic Retinol Bound Protein or Cytosolic Retinoic Acid Bound Protein), and then forming a complex that allows its passage into the cell nucleus. Once inside the nucleus, RET binds to the receptors RAR or RXR, forming a complex operable in binding to RARE (Responsive Element Retinoic Acid). RARE are part of the DNA and represents the promoter region of the target gene, thus, controlling its transcription. Besides this mechanism of action, RET acts in an indirect way without binding to the DNA.

RETs are among the most prescribed and recommended agents in dermatologic therapy. Benefits such as cellular regeneration, exfoliation, and collagen synthesis indicate the importance of RETs in the prevention and treatment of several skin imperfections. Biological effects of RET may include improvement of fine lines, of acne, increased smoothness, improvement of actinic keratose, reduction of hyperpigmentation, among others.

However, there are side effects and other problems in using classic retinoids, and much research has taken place by the cosmetic industry to find alternative, active ingredient to the retinoids that are commonly used in conventional dermatology.

“Skin-Conditioning Agents,” as used herein, means cosmetic ingredients used to create special effects on skin. This group includes substances believed to enhance the appearance of dry or damaged skin and substantive materials which adhere to the skin to reduce flaking and restore suppleness.

“Dermatologically acceptable,” as used herein, means that the compositions or components described are suitable for use in contact with human keratinous tissue without undue toxicity, incompatibility, instability, allergic response, and the like.

“Effective amount,” as used herein, means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

“Improving skin condition,” as used herein, means affecting a visually and/or tactilely perceptible positive change, or benefit, in skin appearance and feel. Benefits that may be provided include, but are not limited to, one or more of the following: Reducing the appearance of wrinkles and coarse deep lines, fine lines, crevices, bumps, and large pores; thickening of keratinous tissue; skin lightening; preventing loss of skin elasticity, for example, due to loss, damage and/or inactivation of functional skin elastin, resulting in such conditions as elastosis, sagging, loss of skin recoil from deformation; change in coloration to the skin, for example, under-eye circles, blotchiness (e.g., uneven red coloration due to, for example, rosacea), sallowness, discoloration caused by hyperpigmentation, etc.

“Signs of skin aging,” as used herein, include, but are not limited to, all outward visibly and tactilely perceptible manifestations, as well as any macro- or micro-effects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including under-eye circles); blotchiness; sallowness; hyper-pigmented skin regions such as age spots and freckles; collagen breakdown.

“Hyperpigmentation,” as used herein, refers to an area of skin wherein the pigmentation is greater than that of an adjacent area of skin (e.g., a pigment spot, an age spot, and the like).

“Derivatives,” as used herein, includes, but is not limited to, amide, ether, ester, amino, carboxyl, acetyl, and/or alcohol derivatives of a given compound.

“Isomers,” as used herein, is understood to include D-isomers, L-isomers, and/or DL-isomers of a given compound.

“Salts,” as used herein, includes, but is not limited to, sodium, potassium, calcium, ammonium, manganese, copper, and/or magnesium salts of a given compound.

“Extract,” as used herein, means material that has been extracted from a plant.

“Antioxidants,” as used herein, means ingredients employed in cosmetics to prevent or retard product spoilage from rancidity (or deterioration from reaction with oxygen). Antioxidants play a vital role in maintaining the quality, integrity, and safety of cosmetic products. Typical cosmetic antioxidants include reducing agents and free radical scavengers.

“Film Formers,” as used herein, means materials which produce a continuous film on skin. These films are used in cosmetics for diverse purposes (e.g., in forming facial masks).

“Skin-Conditioning Agents—Emollient,” as used herein, means cosmetic ingredients which help to maintain the soft, smooth, and pliable appearance of skin. Emollients function by their ability to remain on the skin surface or in the stratum corneum to act as lubricants, to reduce flaking, and to improve the skin's appearance.

“Surfactants—Emulsifying Agents,” “Surfactants,” and “Emulsifying Agents,” as used herein, are employed in cosmetics to prepare emulsions. The efficacy of emulsifying agents depends on their ability to reduce surface tension, to form complex films on the surface of emulsified droplets, and to create a repulsive barrier on emulsified droplets to prevent their coalescence. Emulsion Stabilizers and Viscosity-Increasing Agents can be used as auxiliary emulsifiers to facilitate the process of emulsification or to retard physical changes in emulsions throughout their shelf-life.

“Humectants,” as used herein, means ingredients used in cosmetic products to retard moisture loss from the product during use. This function is generally performed by hygroscopic materials. The efficacy of humectants depends to a large extent on the ambient relative humidity. Humectants which are used for moisturizing skin may be listed separately under Skin-Conditioning Agents—Humectants.

“Skin-Conditioning Agents—Occlusive,” as used herein, means cosmetic ingredients which retard the evaporation of water from the skin surface. By blocking the evaporative loss of water, occlusive materials increase the water content of skin. Occlusive agents are generally lipids which tend to remain on the skin surface. Occlusive is frequently attributed to Skin-Conditioning Agents—Emollient, Skin-Conditioning Agents—Humectant, and Skin-Conditioning Agents—Miscellaneous, which sometimes exhibit occlusive properties upon application to the skin.

“Viscosity Increasing Agents,” as used herein, means and are used to thicken the aqueous portions of cosmetic products. Their ability to perform this function is related to their water solubility or hydrophilic nature. They are widely employed in various types of emulsions. Related functions include Binders, Film Formers, and Emulsion Stabilizers.

“Chelating Agents,” also called “sequestrates,” as used herein means ingredients that have the ability to complex with and inactivate metallic ions in order to prevent their adverse effects on the stability or appearance of cosmetic products. At times it is important to complex calcium or magnesium ions which are incompatible with a variety of cosmetic ingredients. Chelation of ions, for example iron or copper, helps retard oxidative deterioration of finished products.

“Aliphatic,” as used herein, is understood to include both saturated and unsaturated, straight chain (i.e., unbranched) and branched, acyclic, cyclic or polycyclic aliphatic hydrocarbons, which are optionally substitute with one or more functional groups. As will be appreciated by one of ordinary skill in the art, “Aliphatic,” is intended to include, but is not limited to, alkyl, alkenyl, alkynyl, cycloalkyl, cycloalkenyl and cycloalkynyl moieties. “Alkyl,” as used herein, is understood to include straight, branched and cyclic alkyl groups. Further, “Alkyl,” as used herein, is understood to include substituted and unsubstituted groups. Analogous conventions apply to other generic terms such as “alkenyl,” “alkynyl,” and the like.

“Alkenyl,” as used herein, is understood to include a monovalent group derived from a straight- or branched chain hydrocarbon moiety containing at least one carbon-carbon double bond by removal of a single hydrogen atom.

“Anti-Aging,” as used herein, is understood to include promotion of wound healing, protection against ultraviolet radiation damage (e.g., UVA and UVB rays), protection against oxidative stress and/or reduces collagenase production.

“Anti-Inflammatory,” as used herein, is understood to include protection against chemical induced pro-inflammatory cytokine release, protection against bacterial induced (e.g., toll-like receptor-2 (TLR2)) pro-inflammatory cytokine release, protection against metal induced (e.g., toll-like receptor-4 (TLR4)) pro-inflammatory cytokine release, protection against T-cell induced pro-inflammatory cytokine release, protects against ultraviolet induced pro-inflammatory cytokine release and/or reduction of COX expression.

“Anti-Acne,” as used herein, is understood to include reduction in inflammatory lesions, reduction in microcomedone counts and/or reduction in sebum levels.

“Anti-Microbial,” as used herein, is understood to include protection against Propionibacterium acnes (P. acnes) and/or Streptococcus pyogenes (S. pyogenes).

“Enantiomers,” as used herein, are understood to include pairs of chemical compounds whose molecular structures have a nonsuperimposable mirror-image relationship to one another.

“Esters,” as used herein, are understood to include any and all compounds formed when a carboxylic acid and an alcohol combine and release water.

“Hydrates,” as used herein, are understood to include any and all compounds containing chemically combined water in the form of H₂O molecules.

“Solvates,” as used herein, are understood to include any and all compounds formed by the aggregation of one or more solvent molecules with a solute molecule or ion.

“Polymorphs,” as used herein, are understood to include any and all crystal forms of a chemical compound that exhibit polymorphism (i.e., two or more chemically identical but crystallography distinct forms).

Ingredients

The compositions disclosed herein may include one or more of the following ingredients, extracts, fractions thereof, salts, isomers, and/or derivatives thereof and, but is in no way a limitation to other ingredients. Additionally, some ingredients are described by the name required by the International Nomenclature of Cosmetic Ingredients (INCI) while others are described by trade name that comprises one or more INCI-termed ingredients.

In an embodiment, an isoprenylcysteine may be used as an Anti-Aging Agent, an Anti-Inflammatory Agent, an Anti-Acne Agent and/or an Anti-Microbial Agent. In an embodiment, an isoprenylcyeine may be a compound of formula:

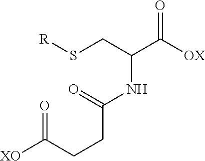

and any variations thereof, including any and all isomers, enantiomers, and potential conversions to salts, esters, polymorphs, hydrates and/or solvates of the isoprenylcysteine, wherein R is an aliphatic group and X is hydrogen, sodium, potassium and/or any other cosmetically acceptable counter-ions including, but not limited to, calcium, ammonium, manganese, copper, magnesium and the like. In certain aspects, an aliphatic group may be C6-C14, C10-C14, C16-C20 and/or C16-C25 hydrocarbons. In other aspects, an aliphatic group may be C12, C16 and/or C20 hydrocarbons.

In certain aspects, an isoprenylcysteine may be N-[1-Carboxy-2-((3,7,11,15,19-pentamethyl-eicos-2-enyl)sulfanyl)-ethyl]succinamic acid or salt of formula:

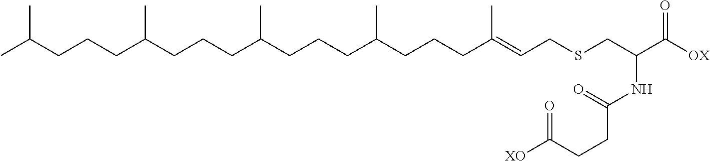

and any variations thereof, including all isomers, enantiomers and potential conversions to salts, esters, polymorphs, hydrates and/or solvates of the N-[1-Carboxy-2-((3,7,11,15,19-pentamethyl-eicos-2-enyl)sulfanyl)-ethyl]succinamic acid or salt.

In other aspects, an isoprenylcysteine may be N-[1-Carboxy-2-((3 ,7, 11,15-tetramethyl-hexadec-2-enyl)sulfanyl)-ethyl]succinamic acid or salt of formula:

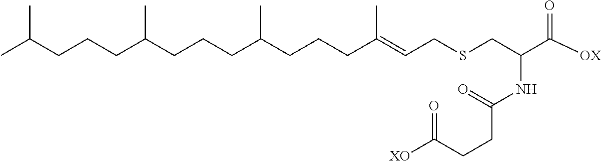

and any variations thereof, including all isomers, enantiomers and potential conversions to salts, esters, polymorphs, hydrates and/or solvates of the N-[1-Carboxy-2-((3,7,11,15-tetramethyl-hexadec-2-enyl)sulfanyl)-ethyl]succinamic acid or salt.

In yet other aspects, an isoprenylcysteine may be N-[1-Carboxy-2-((3,7,11-trimethyl-dodec-2-enyl)sulfanyl)-ethyl]succinamic acid or salt of formula:

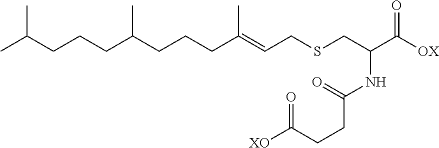

and any variations thereof, including all isomers, enantiomers and potential conversions to salts, esters, polymorphs, hydrates and/or solvates of the N-[1-Carboxy-2-((3,7,11-trimethyl-dodec-2-enyl)sulfanyl)-ethyl]succinamic acid or salt. In certain aspects, the isoprenylcysteine may be Disodium Trimethyldodecenyl Succinoyl Cysteine, Disodium Tetramethylhexadecenyl Succinoyl Cysteine and/or Disodium Pentamethyleicosenyl Succinoyl Cysteine.

However, these non-limiting examples of isoprenylcysteine do not include N-[1-Carboxy-2-(3,7,11-trimethyl-pentadec-2,6,10-trienyl)-2-enylsulfanyl)-ethyl]succinamic acid or salt, or any variations thereof, including any isomers, enantiomers and potential conversions to salts, esters, polymorphs, hydrates and/or solvates of N-[1-Carboxy-2-(3,7,11-trimethyl-pentadec-2,6,10-trienyl)-2-enylsulfanyl)-ethyl]succinamic acid or salt.

In general, isoprenylcysteine compounds are highly active.

In an embodiment, a composition may comprise from about 0.1 w/v % to about 10 w/v % of isoprenylcysteine (and any range or value there between). In certain aspects, a composition may comprise from about 0.1 w/v % to about 7 w/v %, from about 0.1 w/v % to about 3 w/v %, from about 0.1 w/v % to about 1 w/v % or from about 0.1 w/v % to about 0.5 w/v % of isoprenylcysteine (and any range or value there between). In other aspects, a composition may comprise about 0.1 w/v %, about 0.5 w/v %, about 1 w/v % or about 3 w/v % of isoprenylcysteine.

In an embodiment, a composition may comprise from about 0.1% to about 1.5% of isoprenylcystine (and any range or value there between). In other aspects, a composition may comprise about 0.6% or about 0.8% of isoprenylcysteine.

In an embodiment, Aloe Barbadensis Leaf Juice may be useful as a Skin-Conditioning Agent, and for other beneficial properties. Non-limiting, alternatives to Aloe Barbadensis Leaf Juice include various concentrations of aloe. Aloe Barbadensis Leaf Juice may be beneficial to provide hydration. To enrich the hydrating effects, the formulations are created with the pure juice from the leaf of the Aloe Vera Plant. Aloe Vera may provide extra moisture and hydration over other formulations that use water instead of, or as a substitute to, Aloe Vera. Aloe Vera is known to provide many natural moisturizing properties helping to improve the skin's natural firmness, while at the same time helping to keep the skin hydrated.

In certain aspects, a composition may comprise from about 45% to about 75% of Aloe Barbadensis Leaf Juice (and any range or value there between). In other aspects, a composition may comprise about 50% or about 60% of Aloe Barbadensis Leaf Juice.

In an embodiment, Glycerin may be useful as a Skin-Conditioning Agent—Humectant, a Skin Protectant, and for other beneficial properties. In certain aspects, a composition may comprise from about 2% to about 7% Glycerine (and any range or value there between). In other aspects, a composition may comprise about 3.4% Glycerine.

In an embodiment, Caprylic/Capric Triglyceride may be useful as a Skin-Conditioning Agent—Emollient/Occlusive, and for other beneficial properties. In certain aspects, a composition may comprise from about 2% to about 20% of Caprylic/Capric Triglyceride (and any range or value there between). In other aspects, a composition may comprise about 3.5% Caprylic/Capric Triglyceride.

In an embodiment, Dicaprylyl Carbonate may be useful as a Skin-Conditioning Agent—Emollient, and for other beneficial properties. In certain aspects, a composition may comprise from about 0.1% to about 1% of Dicaprylyl Carbonate (and any range or value there between).

In an embodiment, Plumeria Auctifolia Flower Extract may be useful as a Skin-Conditioning Agent, and for other beneficial properties. In certain aspects, a composition may comprise from about 2% to about 6% of Plumeria Auctifolia Flower Extract (and any range or value there between). In other aspects, a composition may comprise about 3% or about 3.4% of Plumeria Auctifolia Flower Extract.

In an embodiment, Glyceryl Stearate and/or PEG-100 Stearate may be used as an Emulsifying Agent, a Skin-Conditioning Agent—Emollient, and for other beneficial properties. In certain aspects, a composition may comprise from about 2% to about 10% of Glyceryl Stearate and/or PEG-100 Stearate (and any range or value there between). In other aspects, a composition may comprise about 2% or about 4% of Glyceryl Stearate and/or PEG-100 Stearate.

In an embodiment, Lexemul™ 68 may be used as an Emulsifying Agent, a Skin-Conditioning Agent—Emollient, and for other beneficial properties. Lexemul™ 68 may comprise from about 50% to about 60% of Glyceryl Stearate and from about 40% to about 50% of PEG-100 Stearate. In certain aspects, a composition may comprise from about 2% to about 10% of Lexemul™ 68 (and any range or value there between). In other aspects, a composition may comprise about 2% or about 4% of Lexemul™ 68.

In an embodiment, Cetyl Alcohol may be used as an Emulsifying Agent, Emulsion Stabilizer, a Viscosity Increasing Agent, and for other beneficial properties. In certain aspects, a composition may comprise from about 0.1 to about 1.5% Cetyl Alcohol (and any range or value there between). in other aspects, a composition may comprise about 0.9% of Cetyl Alcohol.

In an embodiment, Jojoba Esters may be used as a Skin-Conditioning Agent—Emollient, and for other beneficial properties. This botanical ingredient may be used for its moisturizing and softening effects. Light and non-oily, it may add skin conditioning properties to the formulation. Even though jojoba is referred to as oil, it is actually not oil, but is a pure natural moisturizer. It contains many good nutrients for the skin such as antioxidants, Vitamin E, and essential fatty acids.

In certain aspects, a composition may comprise from 0.5% to about 2% Jojoba Esters (and any range or value there between).

In an embodiment, Floraesters™ 30 may be used as a Skin-Conditioning Agent—Emollient, and for other beneficial properties. The composition of Floraesters™ 30 may comprise about 99.95% JoJoba Esters and about 0.05% Tocopherol. In certain aspects, a composition may comprise from about 0.5% to about 2% Floraesters™ 30 (and any range or value there between).

In an embodiment, Tetrahexyldecyl Ascorbate may be used as an Antioxidant, a Skin-Conditioning Agent, and for other beneficial properties. In certain aspects, a composition may comprise from about 0.1% to about 1.5% Tetrahexyldecyl Ascorbate (and any range or value there between). In other aspects, a composition may comprise about 0.8% or about 0.9% of Tetrahexyldecyl Ascorbate.

In an embodiment, Sodium Hyaluronate may be used as a Moisturizing Agent, a Skin-Conditioning Agent, and for other beneficial properties. In certain aspects, a composition may comprise from about 0.01 to about 0.1% Sodium Hyaluronate (and any range or value there between).

In an embodiment, Sorbitol may be used as a Skin-Conditioning Agent—Humectant, and for other beneficial properties. In certain aspects, a composition may comprise from about 0.1% to about 1% of Sorbitol (and any range or value there between).

In an embodiment, Bisabolol may be used as an Anti-Inflammatory Agent, a Skin-Conditioning Agent, and for other beneficial properties. Bisabolol extract, as an active botanical ingredient, has long been referred to as a very soothing agent. Often called German Chamomile, it is used in crèmes and gels throughout the cosmetic industry. Many times, it is chosen because it shows signs of helping to help with puffiness.

In certain aspects, a composition may comprise from 0.01% to about 1% of Bisabolol (and any range or value there between).

In an embodiment, Leuconostoc or Radish Root Ferment Filtrate may be used as an Anti-Microbial Agent, a Skin-Conditioning Agent, and for other beneficial properties. In certain aspects, a composition may comprise from about 0.1% to about 1.5% of Leuconostoc or Radish Root Ferment Filtrate (and any range or value there between). In other aspects, a composition may comprise about 0.8% Leuconostoc or Radish Root Ferment Filtrate.

In an embodiment, Lonicera Caprifolium (Honeysuckle) Flower Extract may be used as a Skin-Conditioning Agent, and for other beneficial properties. In certain aspects, a composition may comprise from about 0.1% to about 1.5% of Lonicera Caprifolium (Honeysuckle) Flower Extract (and any range or value there between). In other aspects, a composition may comprise about 0.6% Lonicera Caprifolium (Honeysuckle) Flower Extract.

In an embodiment, Lonicera Japonica (Honeysuckle) Flower Extract may be used as a Skin-Conditioning Agent, and for other beneficial properties. In certain aspects, a composition may comprise from about 0.1% to about 1.5% of Lonicera Japonica (Honeysuckle) Flower Extract (and any range or value there between). In other aspects, a composition may comprise about 0.8% Lonicera Japonica (Honeysuckle) Flower Extract.

In an embodiment, Populus Tremuloides Bark Extract may be used as a Skin-Conditioning Agent, and for other beneficial properties. In certain aspects, a composition may comprise from about 0.1% to about 1% of Populus Tremuloides Bark Extract (and any range or value there between). In other aspects, a composition may comprise about 0.5% Populus Tremuloides Bark Extract.

In an embodiment, Gluconolactone may be used as an Anti-Acne Agent, a Chelating Agent, Skin-Conditioning Agent, and for other beneficial properties. In certain aspects, a composition may comprise about 0.1% to about 0.5% of Gluconolactone (and any range or value there between). In other aspects, a composition may comprise about 0.3% Gluconolactone.

In an embodiment, NatPres™ Base may be used as an Anti-Acne Agent, a Chelating Agent, Skin Conditioning Agent, and for other beneficial properties. NatPres™ Base may comprise from about 60% to about 70% of Leuconostoc or Radish Root Ferment Filtrate, from about 20% to about 25% of Lonicera Caprifolium (Honeysuckle) Flower Extract, from about 1% to about 20% of Lonicera Japonica (Honeysuckle) Flower Extract, from about 1% to about 20% of Populus Tremuloides Bark Extract, and from about 0.01% to about 10% of Gluconolactone. In certain aspects, a composition may comprise from 0.1% to about 5% of NatPres™ Base (and any range or value there between).

In an embodiment, Avena Sativa (Oat) Kernel Extract may be used as an Antioxidant, a Skin-Conditioning Agent—Emollient, and for other beneficial properties. In certain aspects, a composition may comprise from about 0.1% to about 1% (and any range or value there between).

In an embodiment, Camellia Sinensis (Green Tea) Leaf Extract may be used as an Antioxidant, a Skin-Conditioning Agent, and for other beneficial properties. Camellia Sinensis (Green Tea) Leaf Extract comprises polyphenols. Polyphenols have shown powerful antioxidant activity. When used in cosmetics, it is indicated tea extracts can help and assist with the prevention of collagen breakdown and help improve elasticity—an important factor in maintaining youthful and healthy looking skin.

In certain aspects, a composition may comprise from about 0.01% to about 0.1% Camellia Sinensis (Green Tea) Leaf Extract (and any range or value there between).

In an embodiment, Santalum Album (Sandalwood) Extract may be used as a Skin-Conditioning Agent, and for other beneficial properties. In certain aspects, a composition may comprise from about 0.01% to about 0.5% of Santalum Album (Sandalwood) Extract (and any range or value there between).

In an embodiment, Phellodendron Amurense (Bark) Extract may be used as a Skin-Conditioning Agent, and for other beneficial properties. In certain aspects, a composition may comprise from about 0.01% to about 0.5% of Phellodendron Amurense (Bark) Extract (and any range or value there between).

In an embodiment, Hordeum Distichon (Barley) Extract may be used as a Skin-Conditioning Agent, and for other beneficial properties. In certain aspects, a composition may comprise from about 0.01% to about 1.0% of Hordeum Distichon (Barley) Extract (and any range or value there between).

In an embodiment, a Bois™ 11 may be used as a Skin-Conditioning Agent, and for other beneficial properties. Bois™ 11 may comprise from about 15% to about 25% of Santalum Album (Sandalwood) Extract, from about 15% to about 25% of Phellodendron Amurense (Bark) Extract and from about 50% to about 70% of Hordeum Distichon (Barley) Extract. In certain aspects, a composition may comprise from about 0.1% to about 1% of Bois™ 11 (and any range or value there between).

In an embodiment, Tetrapeptide-30 may be used as a Skin Protectant, a Skin-Conditioning Agent, and for other beneficial properties. Tetrapeptide-30 is peptides that may be selected to topically help with improving the appearance and complexion of healthy skin. Age, environmental stress and harsh conditions often leave uneven skin tones, discoloration and less than youthful skin. This peptide is believed to encourage the skin in maintaining its' own natural vitality.

In certain aspects, a composition may comprise from about 0.1% to about 1% of Tetrapeptide-30 (and any range or value there between).

In an embodiment, Palmitoyl Dipeptide-5 may be used as a Skin-Conditioning Agent, and for other beneficial properties. In certain aspects, a composition may comprise from about 0.1% to about 1% of Palmitoyl Dipeptide-5 (and any range or value there between).

In an embodiment, Diaminobutyroyl Hydroxythreonine may be used as a Skin-Conditioning Agent, and for other beneficial properties. In certain aspects, a composition may comprise from about 0.1% to about 1% of Diaminobutyroyl Hydroxythreonine (and any range or value there between).

In an embodiment, Palmitoyl Dipeptide-5 Diaminohydroxybutyrate may be used as a Skin-Conditioning Agent, and for other beneficial properties. In certain aspects, a composition may comprise from about 0.1% to about 1% of Palmitoyl Dipeptide-5 Diaminohydroxybutyrate(and any range or value there between).

In an embodiment, Syn-Tack™ may be used as a Skin-Conditioning Agent, and for other beneficial properties. Syn-Tack™ may comprise from about 0.01% to about 10% of Palmitoyl Dipeptide-5, Diaminobutyroyl Hydroxythreonine and Diaminobutyroyl Hydroxythreonine, and from about 90% to about 99.99% water. Palmitoyl Dipeptide-5 and Diaminobutyroyl Hydroxythreonine is a preservative-free (patented) peptide combination for delivering ingredients to help with the appearance of radiant skin. In certain aspects, a composition may comprise from about 0.1% to about 1% of Syn-Tack™ (and any range or value there between).

In an embodiment, Butyroyl Pentapeptide-24 may be used as a Skin-Conditioning Agent, and for other beneficial properties. In certain aspects, a composition may comprise from about 0.1% to about 1% of Butyroyl Pentapeptide-24 (and any range or value there between).

In an embodiment, Hydrogenated Lecithin may be used as an Emulsifying Agent, a Skin-Conditioning Agent—Emollient, and for other beneficial properties. Hydrogenated Lecithin is believed to support healthy and natural skin renewal. The look and appearance of the skin is constantly being affected by the skin cell function. The continuous cell renewal process aids in the look and appearance of a healthy, beautiful complexion. Keeping the skin supported during vital renewal periods is an important step.

In certain aspects, a composition may comprise from about 0.1% to about 1% Hydrogenated Lecithin (and any range or value there between).

In an embodiment, Bidens Pilosa Extract may be used as a Skin-Conditioning Agent—Humectant, and for other beneficial properties. Bidens Pilosa Extract may be used for purposes of skin renewal. This botanical plant has been shown clinically to help prevent the formation of early lines and wrinkles. Bidens pilosa is an herbaceous plant having retinoid-like activity. Due to the phytochemical composition of Bidens pilosa, especially by the presence of phytochemical compounds, it supports indications of antioxidant and anti-inflammatory functions and activity on retinoid receptors.

In certain aspects, a composition may comprise from about 0.1% to about 1.5% of Bidens Pilosa Extract (and any range or value there between). In other aspects, a composition may comprise about 0.9% or about 1% of Bidens Pilosa Extract.

In an embodiment, Mauritia Flexuosa Fruit Oil may be used as an Antioxidant, a Moisturizing Agent, a Skin-Conditioning Agent, and for other beneficial properties. Mauritia Flexuosa Fruit Oil may be useful in skin hydration. The nutrients found in the Mauritia Flexuosa Fruit Oil helps to provide natural moisturizing and hydrating properties to help skin maintain a smoother and softener look as a result of better hydration and good care. Mauritia Flexuosa Fruit Oil is oil that may be extracted from the Buriti plant or more specifically the Buriti fruit pulp. It is a rich source of carotenoids, tocopherols, Vitamin-A, monounsaturated fatty acids, and is known for being a powerful natural antioxidant.

In certain aspects, a composition may comprise from about 0.1% to about 1% of Mauritia Flexuosa Fruit Oil (and any range or value there between). In other aspects, a composition may comprise about 0.5% or about 0.8% of Mauritia Flexuosa Fruit Oil.

In an embodiment, Elaeis Guineensis (Palm) Oil may be used as a Skin-Conditioning Agent—Emollient/Humectant, and for other beneficial properties. In certain aspects, a composition may comprise from about 0.1% to about 2% (and any range or value there between). In other aspects, a composition may comprise about 0.9% or about 1% of Elaeis Guineensis (Palm) Oil.

In an embodiment, Gossypium Herbaceum (Cotton) Seed Oil may be used as a Skin-Conditioning Agent—Emollient/Humectant/Occlusive, and for other beneficial properties. In certain aspects, a composition may comprise from about 0.1% to about 1.5% (and any range or value there between). In other aspects, a composition may comprise about 0.6% or about 0.8% of Gossypium Herbaceum (Cotton) Seed Oil.

In an embodiment, Linum Usitatissimum (Linseed) Seed Oil may be used as a Skin-Conditioning Agent—Emollient/Humectant/Occlusive, and for other beneficial properties. In certain aspects, a composition may comprise from about 0.1% to about 1% of Linum Usitatissimum (Linseed) Seed Oil (and any range or value there between). In other aspects, a composition may comprise about 0.6% or about 0.8% of Linum Usitatissimum (Linseed) Seed Oil.

In an embodiment, SAL-14™ may be used as a Skin Conditioning Agent—Emolient/Humectant/Occlusive, and for other beneficial properties. SAL-14™ may comprise from about 12% to about 28% of Bidens Pilosa Extract, from about 0.2 to about 9.7% of Mauritia Flexuosa Fruit Oil, from about 26% to about 41% of Elaeis Guineensis (Palm) Oil, from about 17% to about 36% of Gossypium Herbaceum (Cotton) Seed Oil, from about 10% to about 20% of Linum Usitatissimum (Linseed) Seed Oil and from about 0.01 to about 0.09% of Tocopherol. In certain aspects, a composition may comprise from about 1% to about 4% of SAL14™ (and any range or value there between). In other aspects, a composition may comprise about 4.21 or about 4.25% of SAL-14™.

In an embodiment, Centella Asiatica Leaf Cell Extract may be used as an Antioxidant, Skin-Conditioning Agent, a Skin Protectant, and for other beneficial properties. Centella Asiatica Leaf Cell Extract is believed to provide many nutrients to the skin with phytosterols, amino acids and polysaccharides, making it a unique and nourishing extract. For centuries, the Centella Asiatica has also been identified and traditionally used for its anti-inflammatory properties. These qualities help the Centella Asiatica to support healthy skin at the same time it works in the toning and tightening of aging skin. It is further believed it can also contribute to the production of fibroblast, the cells that increase collagen.

In certain aspects, a composition may comprise from about 0.001% to about 1% of Centella Asiatica Leaf Cell Extract (and any range or value there between). In other aspects, a composition may comprise about 0.01% or about 0.4% of Centella Asiatica Leaf Cell Extract.

In an embodiment, Squalane may be used as a Skin-Conditioning Agent—Occlusive, and for other beneficial properties. In certain aspects, a composition may comprise from about 0.5% to about 2% of Squalane (and any range or value there between). In other aspects, a composition may comprise about 0.7% or about 1% Squalane.

In an embodiment, Allantoin may be used as a Skin-Conditioning Agent, a Skin Protectant, and for other beneficial properties. In certain aspects, a composition may comprise from about 0.05% to about 1.5% of Allantoin (and any range or value there between). In other aspects, a composition may comprise about 0.75% Allantoin.

In an embodiment, Ubiquinone (also known as CoQ-10) may be used as an Antioxidant, a Skin-Conditioning Agent, and for other beneficial properties. Ubiquinone may have additional beneficial properties. This potent Antioxidant is important in assisting the cells in the building of collagen and elastin. Antioxidants are used in cosmetics to help prevent and slow down the signs of aging, as well as for protection and preservation.

In certain aspects, a composition may comprise from about 0.000% to about 0.1% of Ubiquinone (and any range or value there between). In other aspects, a composition may comprise about 0.01% Ubiquinone.

In an embodiment, Dipotassium Glcryrrhizinate may be used as a Chelating Agent. In certain aspects, a composition may comprise from about 0.1% to about 1% Dipotassium Glcryrrhizinate (and any range or value there between).

In an embodiment, Xanthan Gum may be used as a Binder, an Emulsifying Agent, an Emulsion Stabilizer, a Viscosity Increasing Agent, and for other beneficial properties. In certain aspects, a composition may comprise from about 0.1% to about 1.5% of Xanthan Gum (and any range or value there between). in other aspects, a composition may comprise about 0.8% of Xanthan Gum.

In an embodiment, Disodium Ethylenediaminetetraactetic acid (Disodium EDTA) may be used as a Chelating Agent. In certain aspects, a composition may comprise from about 0.001% to about 1.5% of Disodium EDTA (and any range or value there between). In other aspects, a composition may comprise about 0.6% of Disodium EDTA.

In an embodiment, Tocopherol Acetate may be used as an Antioxidant, a Skin-Conditioning Agent, and for other beneficial properties. In certain aspects, a composition may comprise from about 0.05 to about 1.5% of Tocopherol Acetate (and any range or value there between). In other aspects, a composition may comprise about 0.7% or about 0.8% of Tocopherol Acetate.

In an embodiment, Acrylates/C10-30 Alkyl Acrylate Cross-Polymer may be used as an Emulsion Stabilizer, a Viscosity Increasing Agent, and for other beneficial properties. In certain aspects, a composition may comprise from about 0.1% to about 1% of Acrylates/C10-30 Alkyl Acrylate Cross-Polymer (and any range or value there between).

In an embodiment, Cetearyl Glucoside may be used as an Emulsifying Agent, Skin-Conditioning Agent—Emollient, and for beneficial properties. In certain aspects, a composition may comprise from about 0.1% to about 1% of Cetearyl Glucoside (and any range or value there between).

In an embodiment, Phenoxyethanol may be used as a Preservative. In certain aspects, a composition may comprise from about 0.1 to about 2% (and any range or value there between). In other aspects, a composition may comprise about 0.9% or about 1% of Phenoxyethanol.

In an embodiment, Bontanistat™ PF 64 may be used as a Preservative. Bontanistat™ PF 64 may comprise from about 40% to about 60% of Phenoxyethanol, from about 10% to about 20% of Ethylhexyl Glycerin, from about 20% to about 30% of Caprylyl Glycol and from about 5% to about 10% of Hexylene Glycol. In certain aspects, a composition may comprise from about 0.5 to about 2% of Bontanistat™ PF 64 (and any range or value there between). In other aspects, a composition may comprise about 2% of Bontanistat™ PF 64.

In an embodiment, Sodium Hydroxide may be used as a pH Adjuster. In certain aspects, a composition may comprise from about 0.000% to about 0.1% (and any range or value there between).

In an embodiment, Citric Acid may be used as a pH Adjuster. In certain aspects, a composition may comprise from about 0.000% to about 0.1% (and any range or value there between).

In an embodiment, natural albumen may be used as a Skin-Conditioning Agent, and for other beneficial properties. Natural albumen may help tighten skin. Natural albumen may come in liquid form and is a protein. In use, the natural albumen may help with many signs of aging by aiding tired and stressed skin that often promote the showing of premature lines and wrinkles. Natural albumen may help tighten skin to soften the appearance of fine lines and wrinkles.

In an embodiment, synthetic albumen manufactured by various different methods may be used as a Skin-Conditioning Agent, and for other beneficial properties.

In certain aspects, a composition may comprise from about 5% to about 15% natural albumen and/or man-made albumin (and any range or value there between). In other aspects, a composition may comprise about 10% natural albumen and/or man-made albumin.

Natural albumin or synthetic albumin may be used in a number of different compositions. During the compounding process, according to a non-limiting embodiment provided for illustrative purposes only, the natural albumen is kept in a temperature range of about 30° C. to about 60° C.

In an embodiment, Oryza Sativa (Rice) Bran Oil may be used as a Skin-Conditioning Agent—Emollient, and for other beneficial properties. In certain aspects, a composition may comprise from about 0.1% to about 1% of Oryza Sativa (Rice) Bran Oil (and any range or value there between).

In an embodiment, C14-22 Alcohols may be used as Emulsion Stabilizers. In certain aspects, a composition may comprise from about 0.1% to about 1% of C14-22 Alcohols (and any range or value there between).

In an embodiment, C12-20 Glucoside may be used as Emulsifying Agents. In certain aspects, a composition may comprise from about 0.01% to about 0.5% of C12-20 Glucoside (and any range or value there between).

In an embodiment, Montanov™ 68, which may be supplied by SEPPIC may be used as Emulsifying Agents, Emulsion Stabilizers, and for other beneficial properties. Montanov™ 68 may comprise from about 75% to about 85% of C14-22 Alcohols and from about 15% to about 25% of C12-20 Alkyl Glucoside. In certain aspects, a composition may comprise from about 0.1% to about 1% of Montanov™ 68 (and any range of value there between).

In an embodiment, Cetearyl Alcohol may be used as an Emulsion Stabilizer, Opacifying Agent, Viscosity Increasing Agent, and for other beneficial properties. In certain aspects, a composition may comprise from about 0.1 to about 1.5% of Cetearyl Alcohol (and any range or value there between). In other aspects, a composition may comprise about 0.9% Cetearyl Alcohol.

In an embodiment, Montanov™ L, which may be supplied by SEPPPIC, may be used as an Emulsion Stabilizer, Opacifying Agent, Viscosity Increasing Agent, and for other beneficial properties. Montanov™ L may comprise about 80% Cetearyl Alcohol and about 20% Ceary Glucoside. In certain aspects, a composition may comprise from about 0.1 to about 2% of Montanov™ L (and any range or value there between).

In an embodiment, Ricinus Communis (Castor) Seed Oil may be used as a Skin-Conditioning Agent—Occlusive, and for other beneficial properties. In certain aspects, a composition may comprise from about 1% to about 4% Ricinus Communis (Castor) Seed Oil (and any range or value there between). In other aspects, a composition may comprise about 3% Ricinus Communis (Castor) Seed Oil.

In an embodiment, Octyldodecyl Olivate may be used as a Skin-Conditioning Agent—Emollient/Occlusive, a Viscosity Modifying Agent, and for other beneficial properties. Octyldodecyl Olivate may help in moisturizing skin. Non-limiting alternatives may include true monoi oil, Emollients like esters, shine ingredients like silicones, Dimethicone, cyclomethicone, etc. This moisturizing ester is derived from the olive tree. For centuries, the cosmetic industry has known the benefits and richness the oil from the olive can provide.

In certain aspects, a composition may comprise from about 1% to about 5% of Octyldodecyl Olivate (and any range or value there between). In other aspects, a composition may comprise about 3% Octyldodecyl Olivate.

In an embodiment, Olus Oil (consisting primarily of triglycerides of fatty acids) may be used as a Skin-Conditioning Agent—Emollient, and for other beneficial properties. In certain aspects, a composition may comprise from about 2% to about 9% of Olus Oil (and any range or value there between). In other aspects, a composition may comprise about 3.5% of Olus Oil.

In an embodiment, Chondrus Crispus Powder may be used as a Viscosity Agent. In certain aspects, a composition may comprise from about 0.1% to about 1% of Chondrus Crispus Powder (and any range or value there between).

In an embodiment, Sodium Isostearoyl Lactylate may be used as an Emulsifying Agent, and for other beneficial properties. In certain aspects, a composition may comprise from about 0.5% to about 2% of Sodium Isostearyl Lactylate (and any range or value there between). In other aspects, a composition may comprise about 1% Sodium Isostearoyl Lactylate.

In an embodiment, Polysorbate 20 may be used as an Emulsifying Agent, a Solubilizing Agent, and for other beneficial properties. In certain aspects, a composition may comprise about 2% to about 4% of Polysorbate 20 (and any range or value there between). In other aspects, a composition may comprise about 3.2% Polysorbate 20.

In an embodiment, Caprylyl Glycol may be used as a Skin Conditioning Agent, a Preservative, and for other beneficial propeties. In certain aspects, a composition may comprise from about 0.25% to about 1% of Caprylyl Glycol (and any range or value there between). In other aspects, a composition may comprise about 2% of Caprylyl Glycol.

In an embodiment, Cucumis Sativus (Cucumber) Fruit Extract may be used as an Essential Oil, a Fragrance Ingredient, and for other beneficial properties. In certain aspects, a composition may comprise from about 0.1% to about 2% of Cucumis Sativus (and any range or value there between).

In an embodiment, Glyceryl Caprylate may be used as a Emulsifying Agent, a Skin-Conditioning Agent—Emollient, and for other beneficial properties. In certain aspects, a composition may comprise from about 0.1% to about 0.4% of Glyceryl Caprylate (and any range or value there between). In other aspects, a composition may comprise about 0.5% of Glyceryl Caprylate.

In an embodiment, Hydrolyzed Quinoa may be used as a Skin-Conditioning Agent, a Skin Protectant, and for other beneficial properties. Hydrolyzed Quinoa may help repair skin and is known for its essential nutrients. It is believed Hydrolyzed Quinoa assists in the repair, protection and conditioning of the skin.

In certain aspects, a composition may comprise from about 0.05% to about 0.2% of Hydrolyzed Quinoa (and any range or value there between). In other aspects, a composition may comprise about 0.1% of Hydrolyzed Quinoa.

In an embodiment, Phenylpropanol may be used as a Preservative. In certain aspects, a composition may comprise from about 0.1% to about 0.4% of Phenylpropanol (and any range or value there between). In other aspects, a composition may comprise about 0.5% of Phenylpropanol.

In an embodiment, Dermasoft™ LP, which may be supplied by Prospector, may be used as a Preservative. Dermasoft™ LP may comprise about 50% Caprylyl Glycol, from about 5% to about 10% Glycerin and Glyceryl Caprylate, from about 5% to about 10% Phenylpropanol and from about 1% to about 5% Water. In certain aspects, a composition may comprise from about 0.5% to about 3.6% of Dermasoft™ LP (and any range or value there between). In other aspects, a composition may comprise about 3.60% of Dermasoft™ LP.

In an embodiment, Sodium Salt of Pyroglutamic Acid (Sodium PCA) may be used as a Skin-Conditioning Agent—Humectant, and for other beneficial properties. In certain aspects, a composition may comprise from about 0.1 to about 1% of Sodium PCA (and any range or value there between).

In an embodiment, Stearic Acid may be used as an Emulsifying Agent. In certain aspects, a composition may comprise from about 0.1% to about 1% Stearic Acid (and any range or value there between).

In an embodiment, Tocopherol (aka Vitamin E) may be used as an Antioxidant, a Skin-Conditioning Agent—Emollient/Occlusive, and for other beneficial properties. Tocopherol has been used for generations for its moisturizing, nourishing and protection properties. When used topically, it aids and assists in helping to provide an antioxidant defense for the skin. It is a fat-soluble antioxidant.

In certain aspects, a composition may comprise from about 0.01 to about 0.1 of Tocopherol (and any range or value there between).

It will be appreciated to one of skill in the art that known natural and man-made alternatives to any of the above ingredients may be used in substitution.

Compositions or Complexes

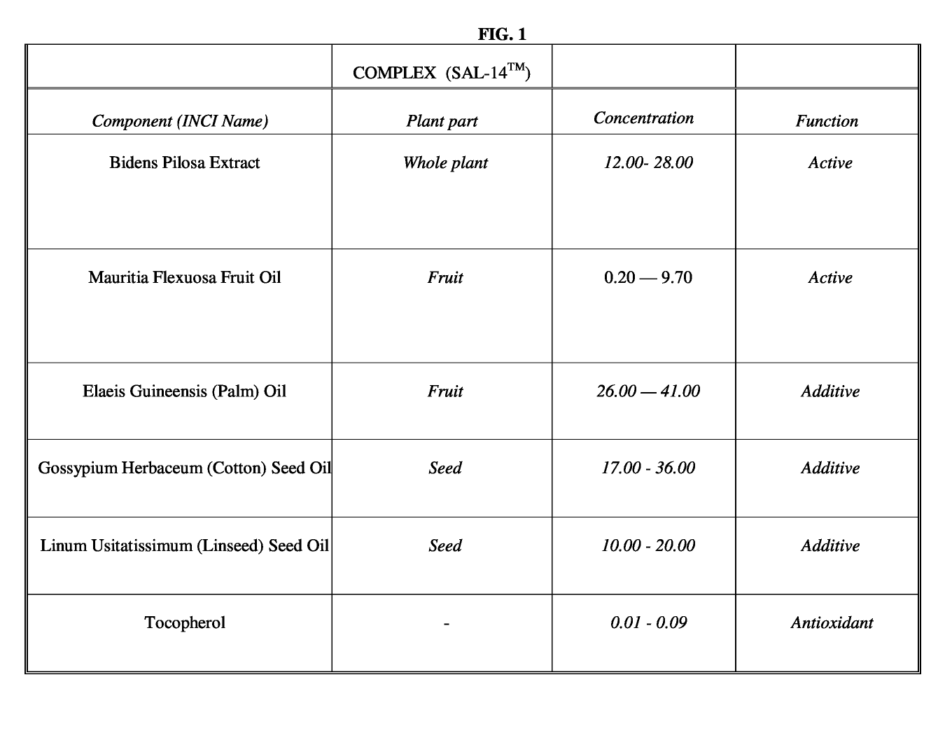

Referring to FIG. 1, an illustrative composition or complex is presented. The complex is a genotoxic compound or at least a DNA composition having protective capacities for cosmetic and pharmaceutical use after sun-induced stress. In an embodiment, the composition or complex comprises: (a) from about 12 w/v % to about 28 w/v % of a Bidens Pilosa Extract (and any range or value there between); (b) from about 0.2 w/v % to about 10 w/v % of Mauritia Flexuosa Fruit Oil (and any range or value there between), (c) from about 26 w/v % to about 41 w/v % of Elaeis Guineensis (Palm) Oil (and any range or value there between); (d) from about 17 w/v % to about 36 w/v % of Gossypium Herbaceum (Cotton) Seed Oil in (and any range or value there between); (e) from about 10 w/v % to about 20 w/v % of Linum Usitatissimum (Linseed) Seed Oil (and any range or value there between); and (f) from about 0.1 w/v % to about 0.09 w/v % of Tocopherol (and any range or value there between). It will be appreciated to one of skill in the art that known natural and man-made alternatives to any of the above ingredients may be used in substitution.

In some aspects, the composition or complex comprises: (a) from about 20 w/v % to about 28 w/v % of Bidens Pilosa Extract (and any range or value there between); (b) from about 5 w/v % to about 10 w/v % of Mauritia Flexuosa Fruit Oil in a concentration (and any range or value there between); (c) from about 26 w/v % to about 35 w/v % of Elaeis Guineensis (Palm) Oil (and any range or value there between); (d) from about 17 w/v % to about 34 w/v % of Gossypium Herbaceum (Cotton) Seed Oil (and any range or value there between); (e) from about 10 w/v % to about 15 w/v % of Linum Usitatissimum (Linseed) Seed Oil (and any range or value there between); and (f) from about 0.01 w/v % to about 0.05 w/v % Tocopherol (and any range or value there between). It will be appreciated to one of skill in the art that known natural and man-made alternatives to any of the above ingredients may be used in substitution.

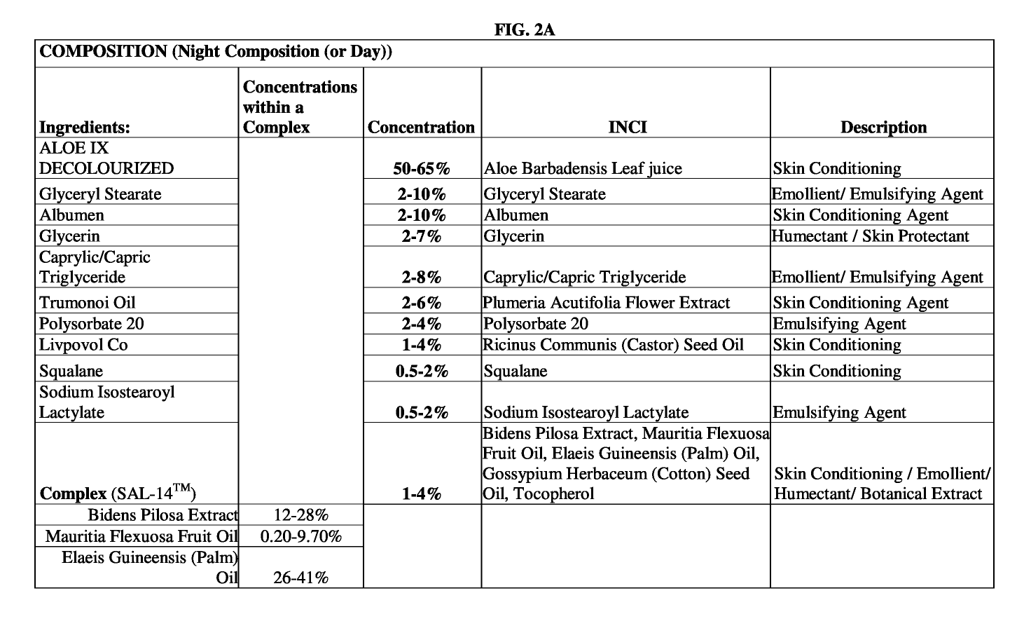

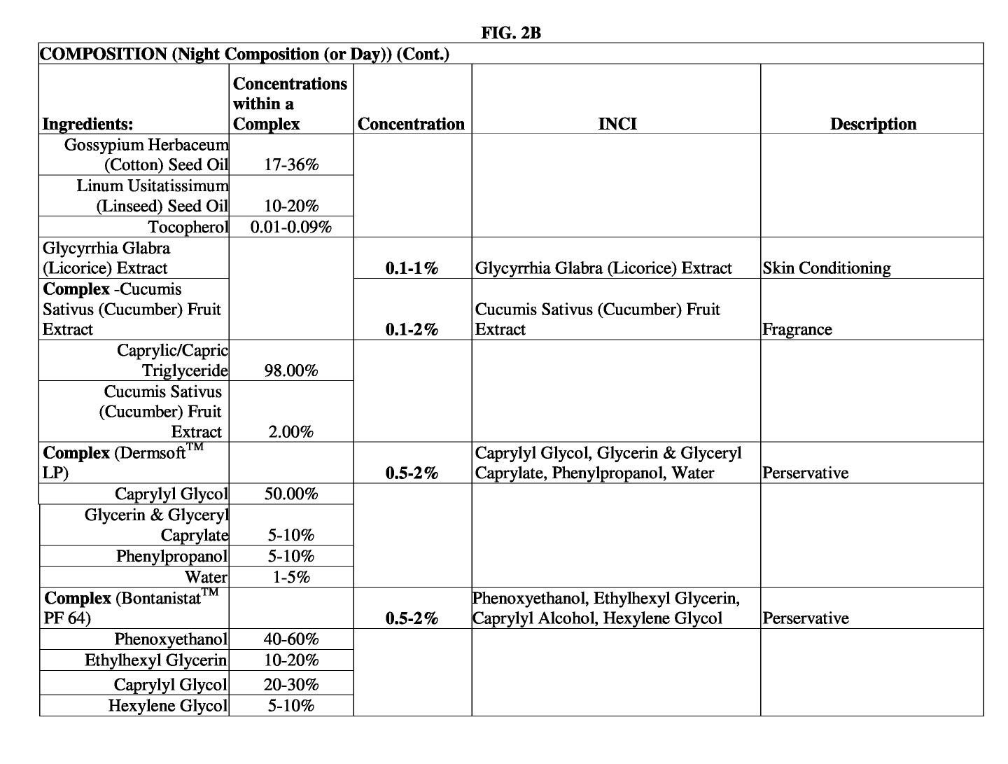

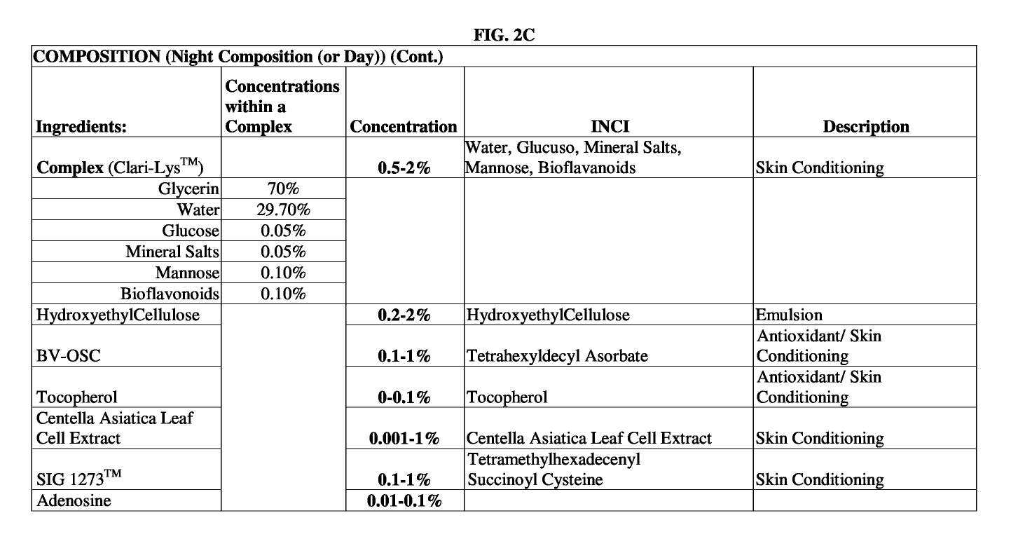

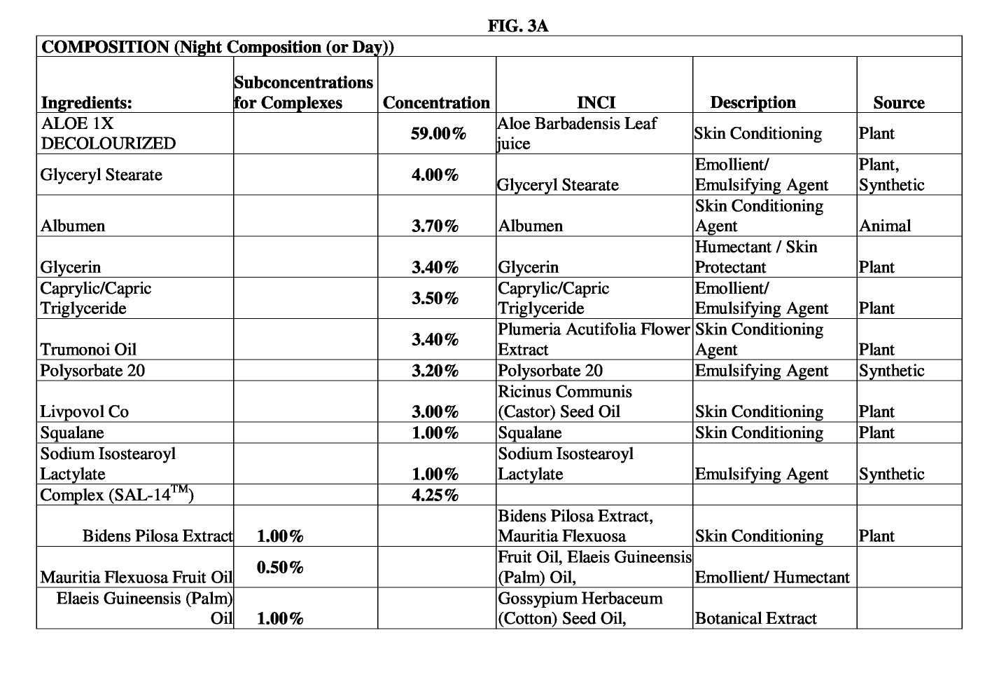

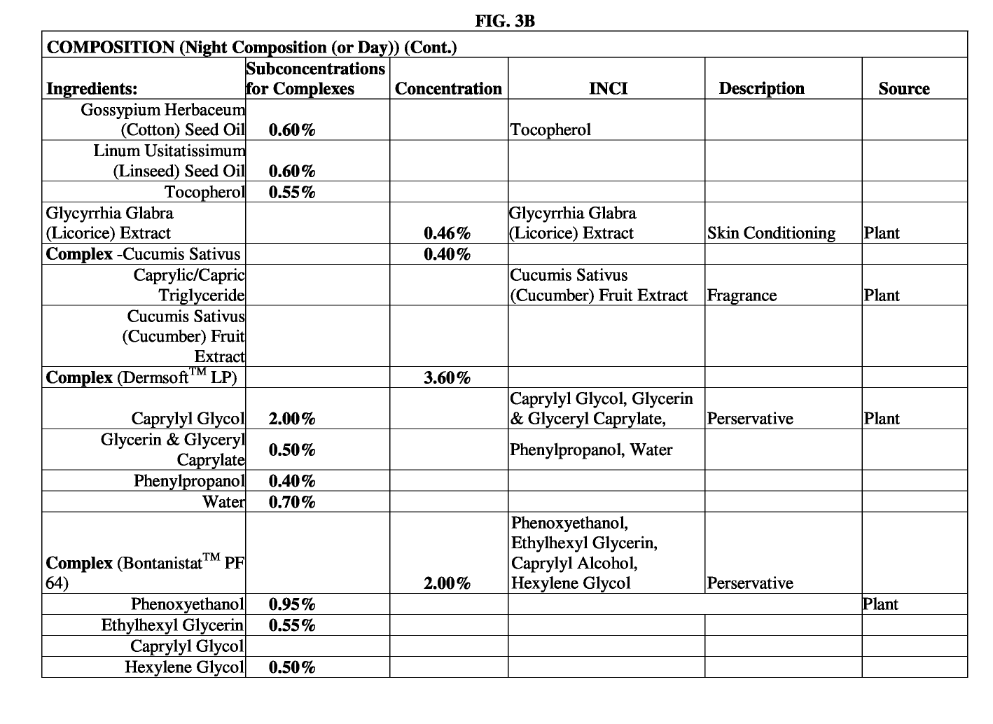

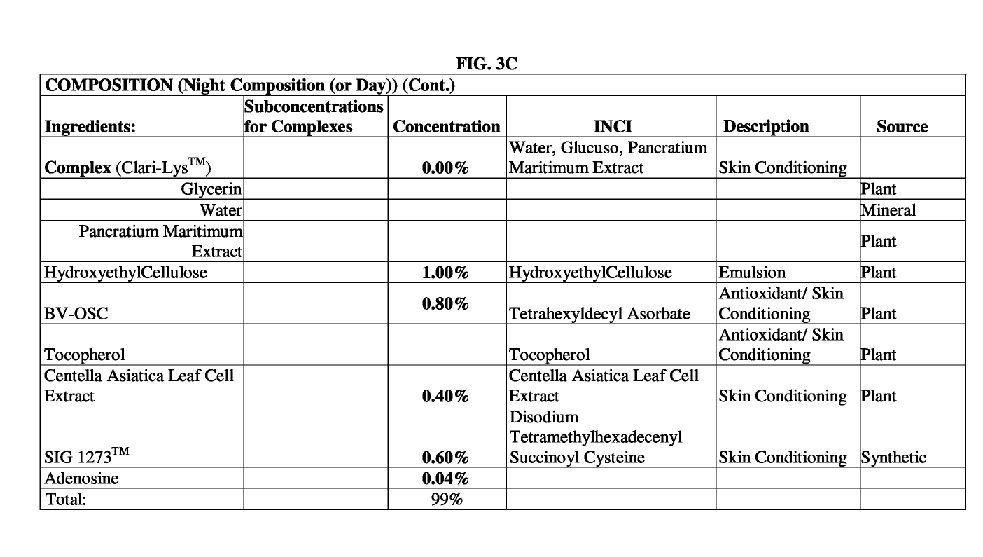

As illustrated in FIGS. 2-3, the composition or complex of FIG. 1 may only comprise, in a non-limiting embodiment, from about 1 w/v % to about 5 w/v % of the respective components illustrated therein (and any range or value there between).

The composition or complex of FIG. 1 may, in some aspects, comprise about 2 w/v % of the components.

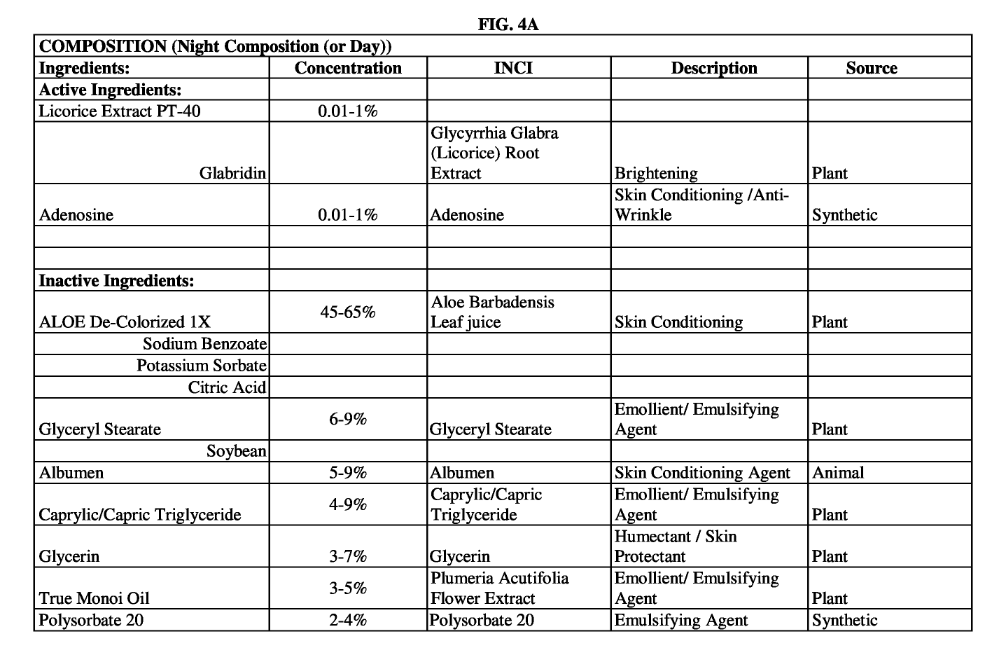

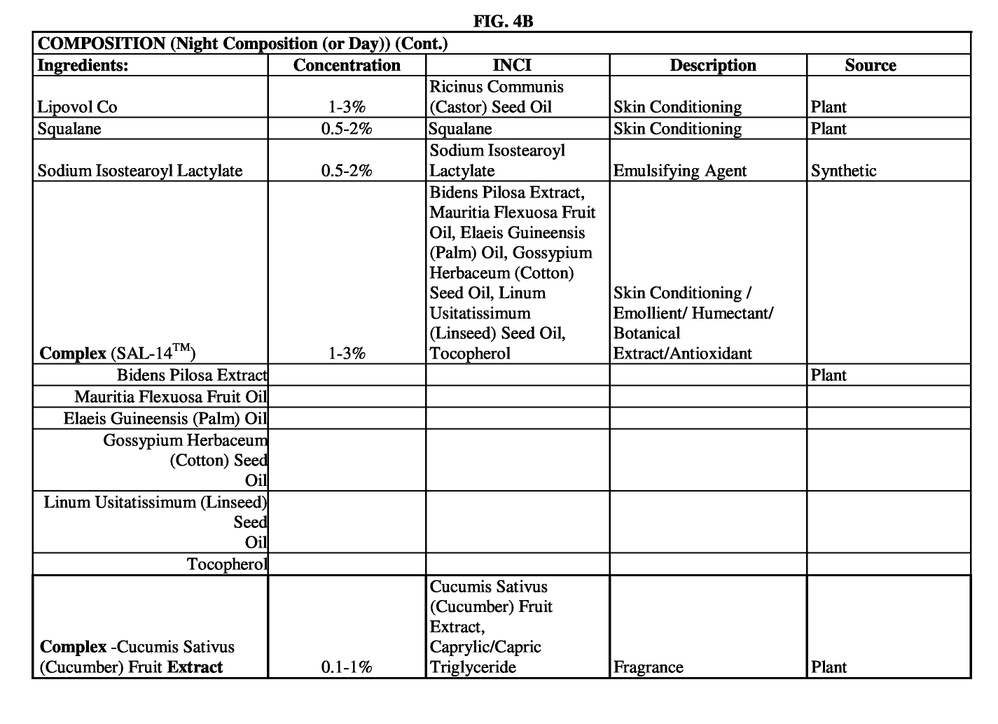

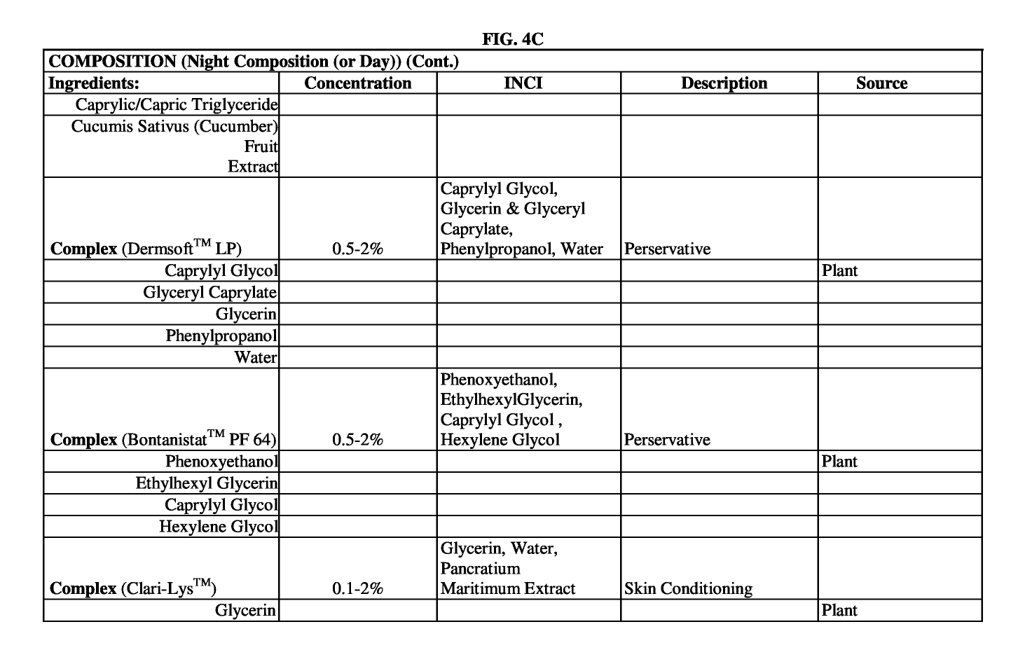

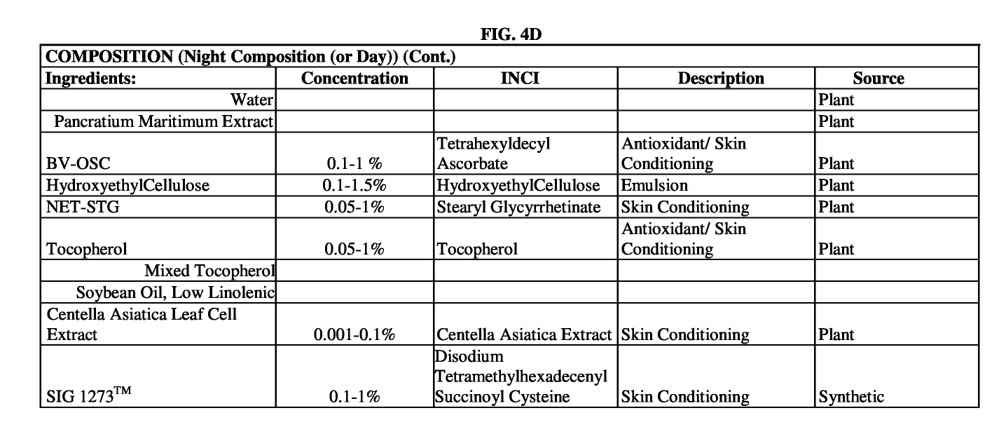

Referring to FIGS. 2-4, a non-limiting illustrative embodiment of a composition, which may be used in the morning or at night, but, preferably, at night, includes a combination, either all or in part, according to ingredient and the contributing percentage of each ingredient. It will be appreciated to one of skill in the art that the percentages provided are for purposes of example only and that the percentages may vary. Further, it will be appreciated to one of skill in the art that known natural or man-made alternatives to any the above ingredients may be used in substitution.

In an embodiment, the composition comprises: Aloe Barbadensis Leaf juice, Glyceryl Stearate, Albumen, Glycerin, Caprylic/Capric Triglyceride, Plumeria Acutifolia Flower Extract, Polysorbate 20, Ricinus Communis (Castor) Seed Oil, Squalane, Sodium Isostearoyl Lactylate, Bidens Pilosa Extract, Mauritia Flexuosa Fruit Oil, Elaeis Guineensis (Palm) Oil, Gossypium Herbaceum (Cotton) Seed Oil, Tocopherol, Glycyrrhia Glabra (Licorice) Extract, Cucumis Sativus (Cucumber) Fruit Extract, Caprylyl Glycol, Glycerin & Glyceryl Caprylate, Phenylpropanol, Water, Phenoxyethanol, Ethylhexyl Glycerin, Caprylyl Alcohol, Hexylene Glycol, Water, Glucuso, Mineral Salts, Mannose, Bioflavanoids, HydroxyethylCellulose, Tetrahexyldecyl Asorbate, Tocopherol, Centella Asiatica Leaf Cell Extract, Disodium Tetramethylhexadecenyl Succinoyl Cysteine, and Adenosine.

In another embodiment, the composition comprises: (a) from about 50% to about 65% of Aloe Barbadensis Leaf juice (and any range or value there between); (b) from about 2% to about 10% of Glyceryl Stearate (and any range or value there between); (c) from about 2% to about 10% of Albumen (and any range or value there between); (d) from about 2% to about 7% of Glycerin (and any range or value there between); (e) from about 2% to about 8% of Caprylic/Capric Triglyceride (and any range or value there between); (f) from about 2% to about 6% of Plumeria Acutifolia Flower Extract (and any range or value there between); (g) from about 2% to about 4% of Polysorbate 20 (and any range or value there between); (h) from about 1% to about 4% of Ricinus Communis (Castor) Seed Oil (and any range or value there between); (i) from about 0.5% to about 2% of Squalane (and any range or value there between); (j) from about 0.5% to about 2% of Sodium Isostearoyl Lactylate (and any range or value there between); (k) from about 1% to about 4% of Complex (including Bidens Pilosa Extract, Mauritia Flexuosa Fruit Oil, Elaeis Guineensis (Palm) Oil, Gossypium Herbaceum (Cotton) Seed Oil, Tocopherol) (and any range or value there between); (1) from about 0.1% to about 1% of Glycyrrhia Glabra (Licorice) Extract (and any range or value there between); (m) from about 0.1% to about 2% of Complex (including Cucumis Sativus (Cucumber) Fruit Extract, Caprylyl Glycol) (and any range or value there between); (n) from about 0.5% to about 2% of Complex (including Glycerin & Glyceryl Caprylate, Phenylpropanol, Water) (and any range or value there between); (o) from about 0.5% to about 2% of Complex (including Phenoxyethanol, Ethylhexyl Glycerin, Caprylyl Alcohol, Hexylene Glycol) (and any range or value there between); (p) from about 0.5% to about 2% of Complex (including Water, Glucuso, Mineral Salts, Mannose, Bioflavanoids) (and any range or value there between); (q) from about 0.2% to about 2% of HydroxyethylCellulose (and any range or value there between); (r) from about 0.1% to about 1% of Tetrahexyldecyl Asorbate (and any range or value there between); (s) from about 0.01% to about 0.1% of Tocopherol (and any range or value there between); (t) from about 0.001% to about 1% of Centella Asiatica Leaf Cell Extract (and any range or value there between); (u) from about 0.1% to about 1% of Disodium Tetramethylhexadecenyl Succinoyl Cysteine (and any range or value there between); and (v) from about 0.01% to about 0.1% of Adenosine (and any range or value there between). It will be appreciated to one of skill in the art that known natural or man-made alternatives to any the above ingredients may be used in substitution.

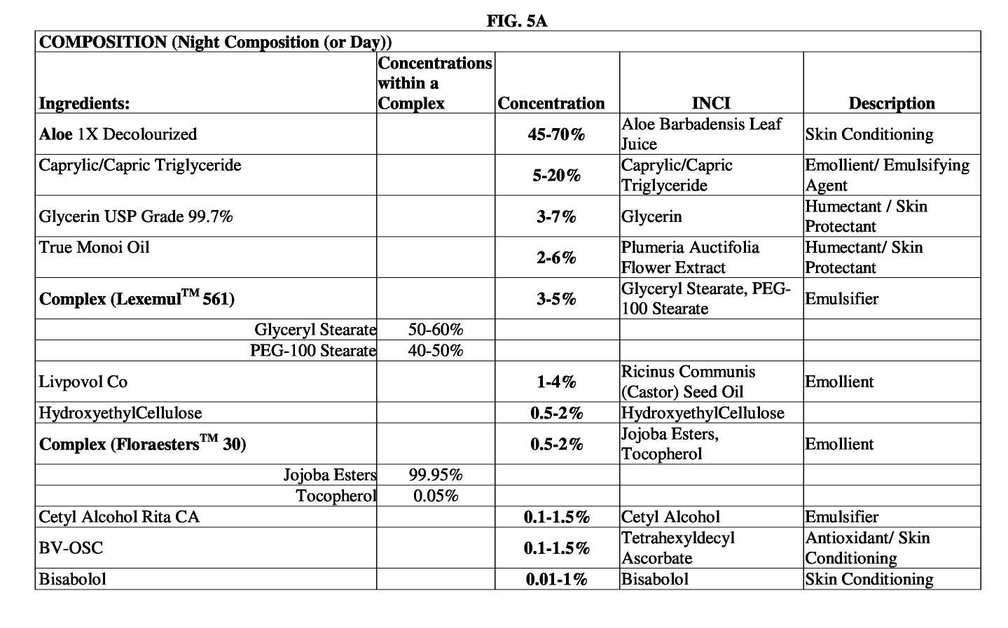

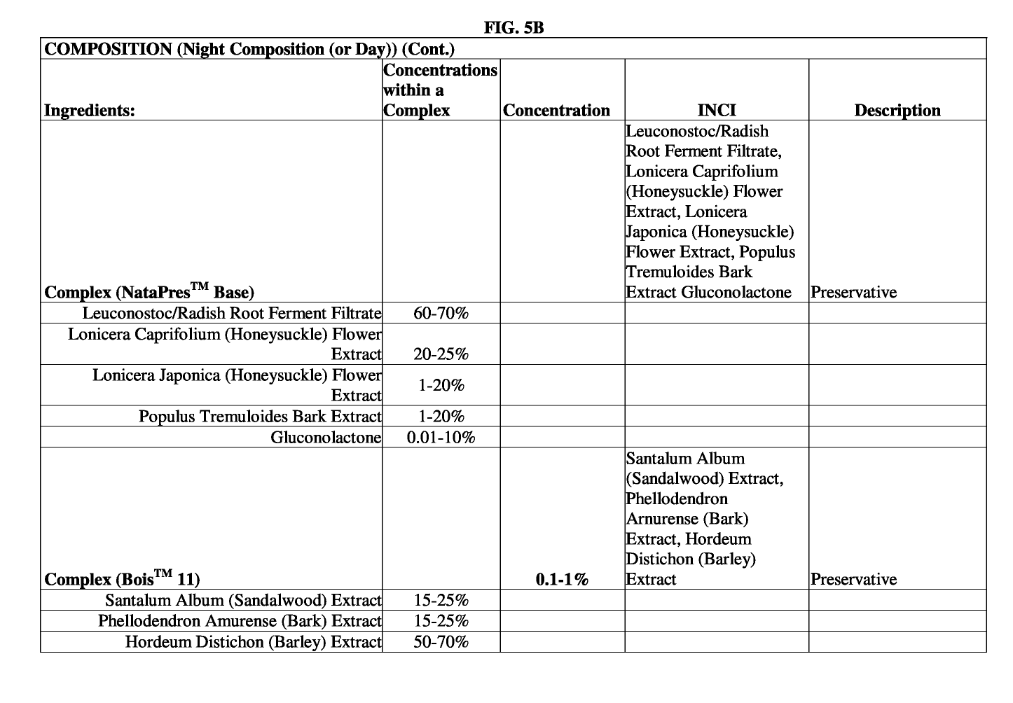

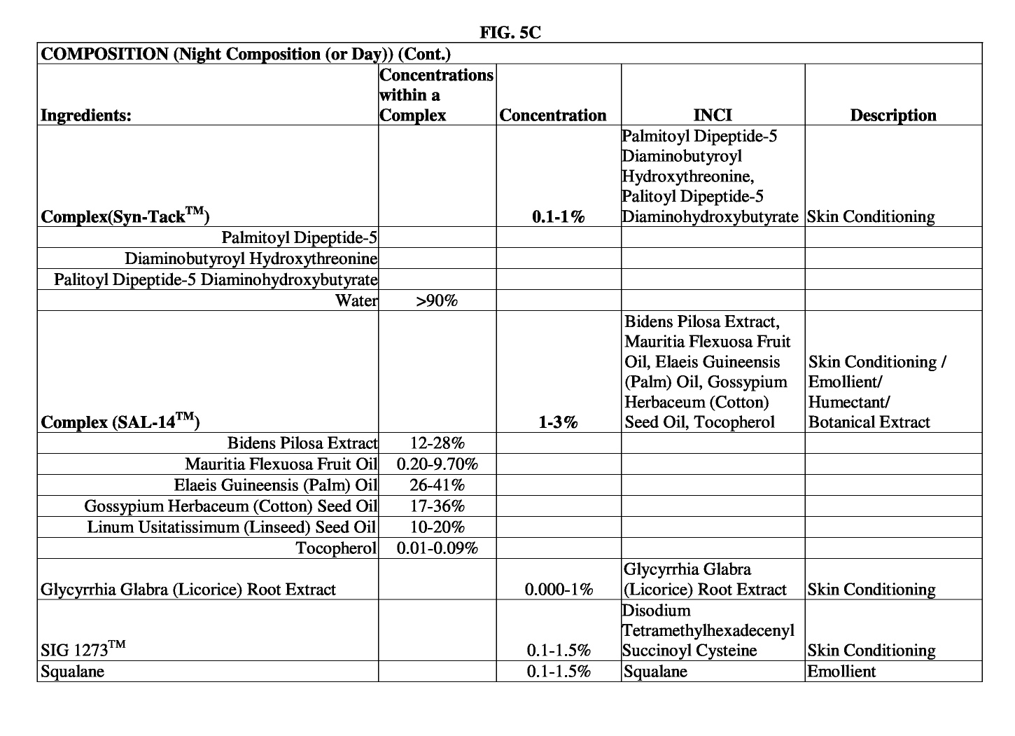

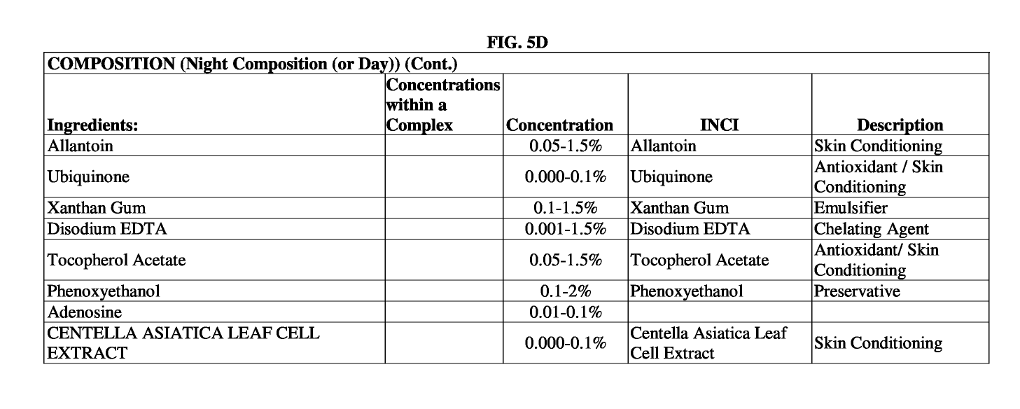

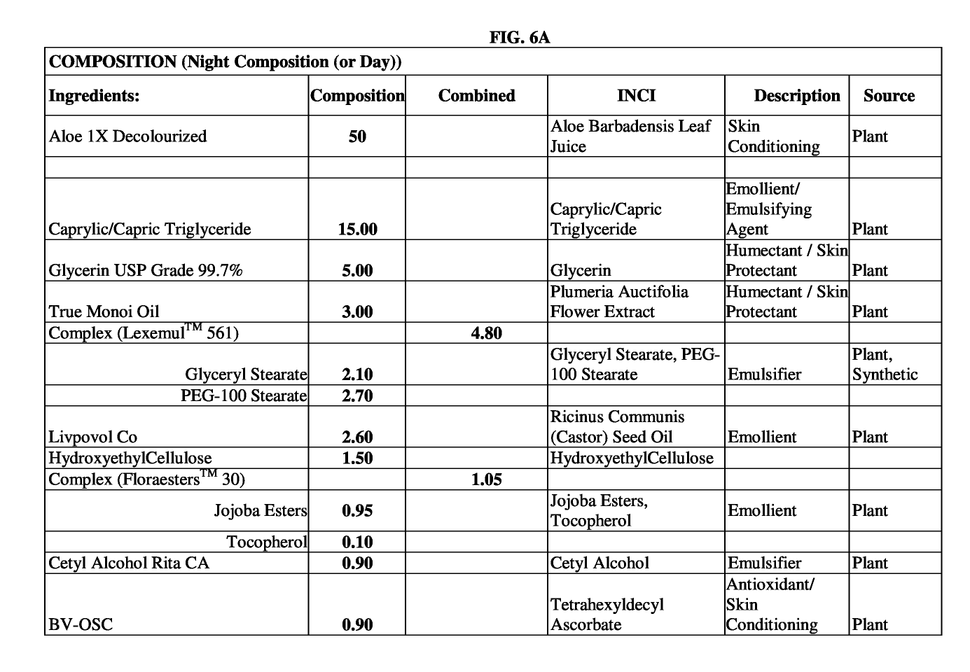

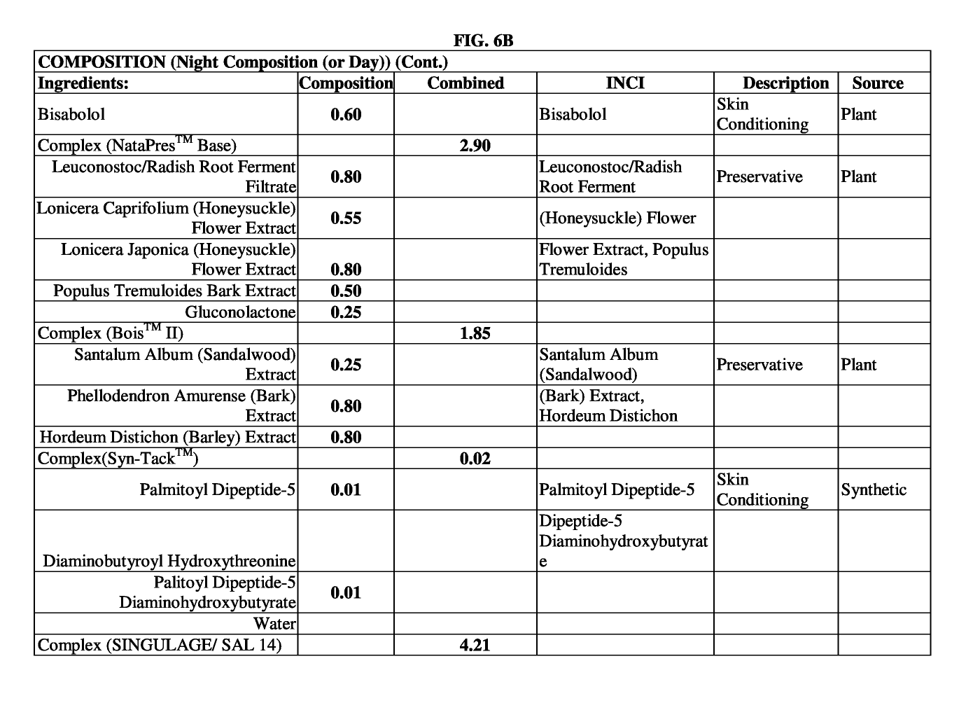

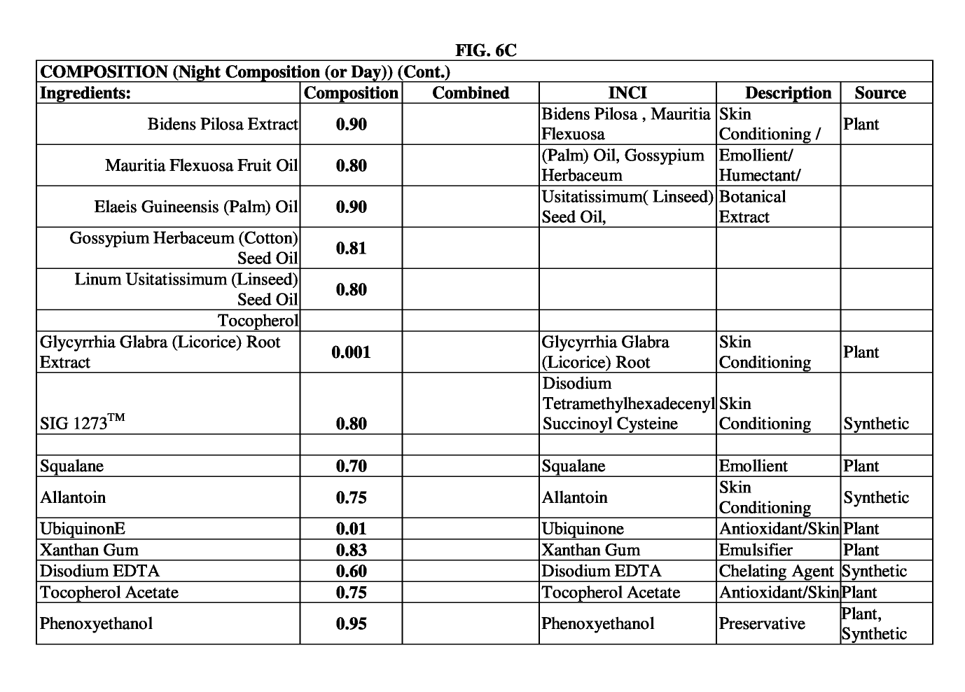

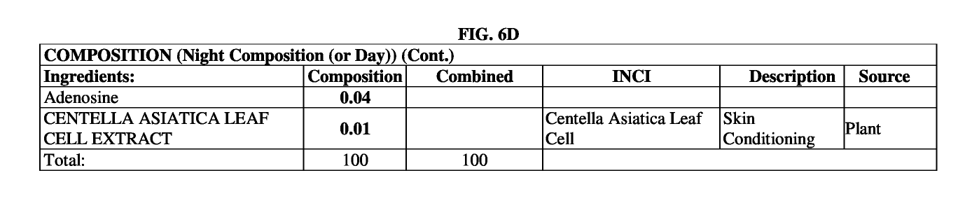

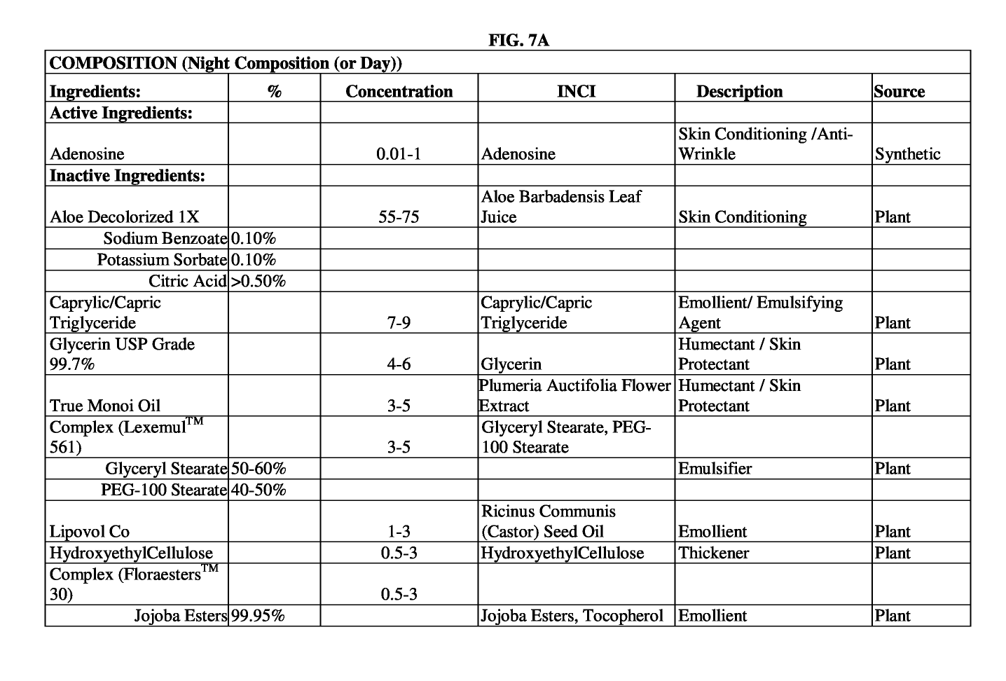

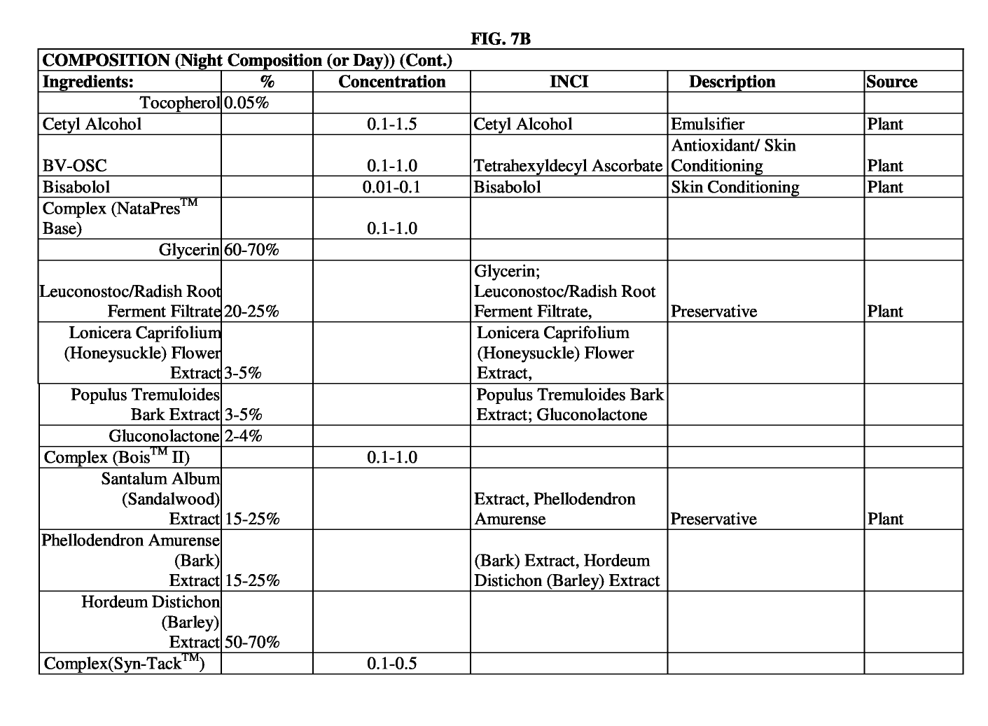

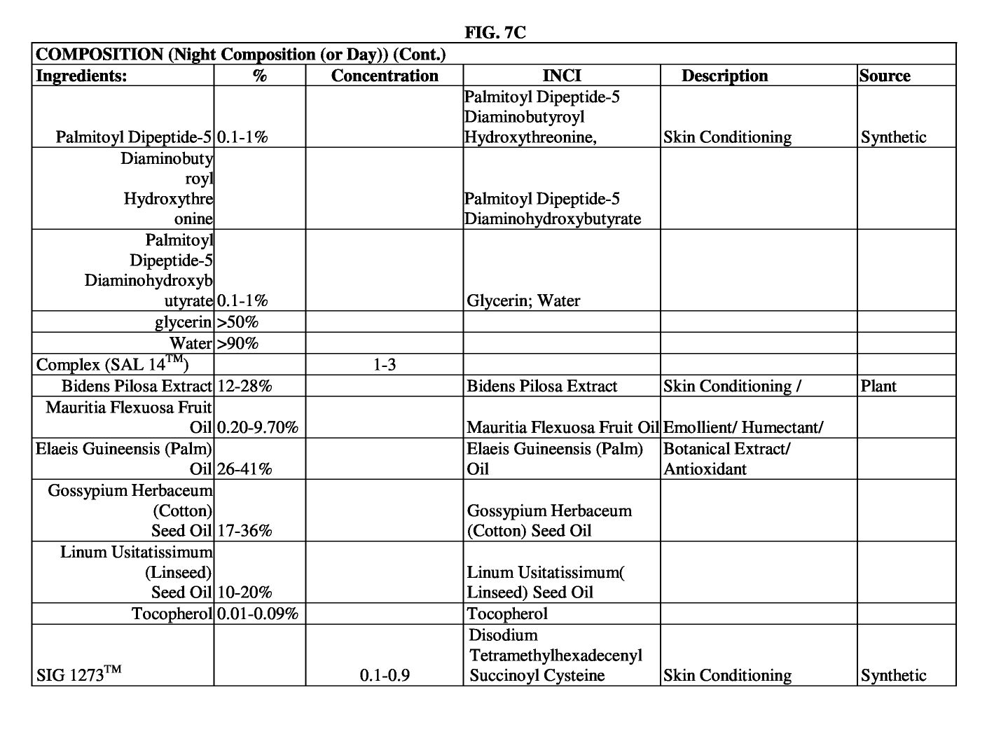

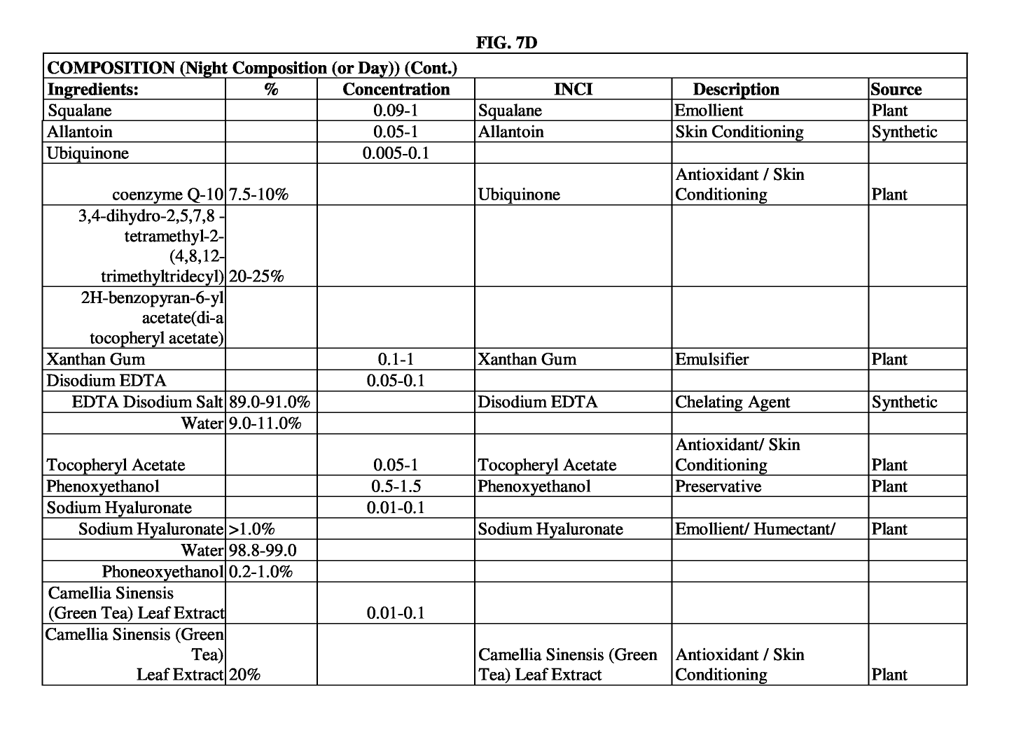

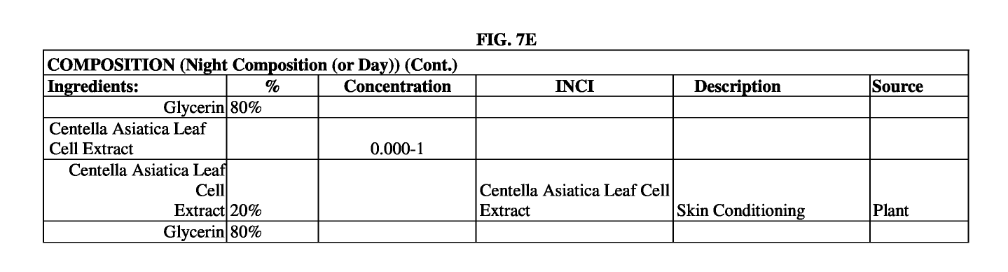

FIGS. 5-7 illustrates an exemplary, non-limiting embodiment, of a composition that may be used either in the morning or at night, but, preferably, in the morning, includes a combination, either all or in part, according to ingredient and the contributing percentage of each ingredient. It will be appreciated to one of ordinary skill that the percentages provided are for purposes of example only and that the percentages may vary and substitute ingredients noted, and known in the art, may be used.

In an embodiment, the composition comprises: (a) from about 45% to about 70% of Aloe Barbadensis Leaf Juice (and any range or value there between); (b) from about 5% to about 20% of Caprylic/Capric Triglyceride (and any range or value there between); (c) from about 3% to about 7% of Glycerine (and any range or value there between); (d) from about 2% to about 6% of Plumeria Auctifolia Flower Extract (and any range or value there between); (e) from about 3% to about 5% of Complex (including Glyceryl Stearate, PEG-100 Stearate) (and any range or value there between); (f) from about 1% to about 4% of Ricinus Communis (Castor) Seed Oil (and any range or value there between); (g) from about 0.5% to about 2% of HydroxyethylCellulose (and any range or value there between); (h) from about 0.5% to about 2% of Complex (including Jojoba Esters, Tocopherol) (and any range or value there between); (i) from about 0.1% to about 1.5% of Cetyl Alcohol (any range or value there between); (j) from about 0.1% to about 1.5% of Tetrahexyldecyl Ascorbate (and any range or value there between); (k) from about 0.01% to about 1% of Bisabolol (and any range or value there between); (1) from about 0.1% to about 1% of Complex (including Leuconostoc/Radish Root Ferment Filtrate, Lonicera Caprifolium (Honeysuckle) Flower Extract, Lonicera Japonica (Honeysuckle) Flower Extract, Populus Tremuloides Bark Extract, Gluconolactone) (and any range or value there between); (m) from about 0.1% to about 1% of Complex (including Santalum Album (Sandalwood) Extract, Phellodendron Amurense (Bark) Extract, Hordeum Distichon (Barley) Extract) (and any range or value there between); (n) from about 0.1% to about 1% of Complex (including Palmitoyl Dipeptide-5 Diaminobutyroyl Hydroxythreonine, Palitoyl Dipeptide-5 Diaminohydroxybutyrate) (and any range or value there between); (o) from about 1% to about 3% of Complex (including Bidens Pilosa Extract, Mauritia Flexuosa Fruit Oil, Elaeis Guineensis (Palm) Oil, Gossypium Herbaceum (Cotton) Seed Oil, Tocopherol) (and any range or value there between); (p) from about 0.000% to about 1% of Glycyrrhia Glabra (Licorice) Root Extract (and any range or value there between); (q) from about 0.1% to about 1.5% of Disodium Tetramethylhexadecenyl Succinoyl Cysteine (and any range or value there between); (r) from about 0.1% to about 1.5% of Squalane (and any range or value there between); (s) from about 0.05% to about 1.5% of Allantoin (and any range or value there between); (t) from about 0.000% to about 0.1% of Ubiquinone (and any range or value there between); (u) from about 0.1% to about 1.5% of Xanthan Gum (and any range or value there between); (v) from about 0.001% to about 1.5% of Disodium EDTA (and any range or value there between); (w) from about 0.05% to about 1.5% of Tocopherol Acetate (and any range or value there between); (x) from about 0.1% to about 2% of Phenoxyethanol (and any range or value there between); (y) from about 0.01% to about 0.1% Adenosine (and any range or value there between); and (z) from about 0.000% to about 0.1% of Centella Asiatica Leaf Cell Extract (and any range or value there between). It will be appreciated to one of skill in the art that known natural and man-made alternatives to any of the above ingredients may be used in substitution.

In the foregoing description of certain embodiments, specific terminology has been resorted to for the sake of clarity. The disclosure, however, is not intended to be limited to the specific terms so selected, and it is to be understood that each specific term includes other technical equivalents that operate in a similar manner to accomplish a similar technical purpose.

In addition, the foregoing describes only some embodiments of the invention(s), and alterations, modifications, additions and/or changes can be made thereto without departing from the scope and spirit of the disclosed embodiments, the embodiments being illustrative and not restrictive.

Furthermore, invention(s) have described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention(s). Also, the various embodiments described above may be implemented in conjunction with other embodiments, e.g., aspects of an embodiment may be combined with aspects of another embodiment to realize yet other embodiments. Further, each independent feature or component of any given assembly may constitute an additional embodiment.

Other Definitions

As used herein, the terms “a,” “an,” “the,” and “said” mean one or more, unless the context dictates otherwise.

As used herein, the term “about” means the stated value plus or minus a margin of error or plus or minus 10% if no method of measurement is indicated.

As used herein, the term “or” means “and/or” unless explicitly indicated to refer to alternatives only or if the alternatives are mutually exclusive.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “containing,” “contains,” and “contain” have the same open-ended meaning as “comprising,” “comprises,” and “comprise,” provided above.

As used herein, the terms “having,” “has,” and “have” have the same open-ended meaning as “comprising,” “comprises,” and “comprise,” provided above.

As used herein, the terms “including,” “includes,” and “include” have the same open-ended meaning as “comprising,” “comprises,” and “comprise,” provided above.

As used herein, the phrase “consisting of” is a closed transition term used to transition from a subject recited before the term to one or more material elements recited after the term, where the material element or elements listed after the transition term are the only material elements that make up the subject.

As used herein, the term “simultaneously” means occurring at the same time or about the same time, including concurrently.

IV. Incorporation By Reference. All patents and patent applications, articles, reports, and other documents cited herein are fully incorporated by reference to the extent they are not inconsistent with this invention.

What is claimed is:

1. A topical composition comprising:
(a) extract of bidens pilosa;
(b) isoprenylcysteine; and
(c) a dermatologically acceptable carrier.

2. The topical composition of claim 1 further comprising mauritia flexuosa fruit oil.

3. The topical composition of claim 1 further comprising elaeis guineessis oil.

4. The topical composition of claim 1 further comprising gossypium herbaceum seed oil.

5. The topical composition of claim 1 further comprising linum usitatissimum seed oil.

6. The topical composition of claim 1 further comprising tocopherol.

7. The topical composition of claim 1 further comprising centella asiatica leaf cell extract.

8. The topical composition of claim 1 further comprising one or more of aloe barbadensis leaf juice, natural albumen, and tocopherol.

9. The topical composition of claim 1 further comprising one or more of jojoba esters, camellia sinensis leaf extract, ubiquinone, bisabolol, and palmitoyl dipeptide-5 diaminobutyroyl hydroxythreonine.

10. The topical composition of claim 1 further comprising pancratium maritimum extract.

11. The topical composition of claim 1 further comprising tetrahexyldecyl ascorbate.

12. The topical composition of claim 1 further comprising camellia sinensis (green tea) leaf extract.

13. The topical composition of claim 1, wherein the isoprenylcysteine is a compound of formula: 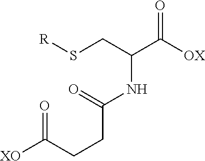
and any variations thereof, including any and all isomers, enantiomers, and potential conversions to salts, esters, polymorphs, hydrates and/or solvates of the isoprenylcysteine, wherein R is an aliphatic group and X is hydrogen, sodium, potassium.

14. The topical composition of claim 1, wherein the isoprenylcysteine is N-[1-Carboxy-2-((3,7,11,15,19-pentamethyl-eicos-2-enyl)sulfanyl)-ethyl]succinamic acid or salt of formula: 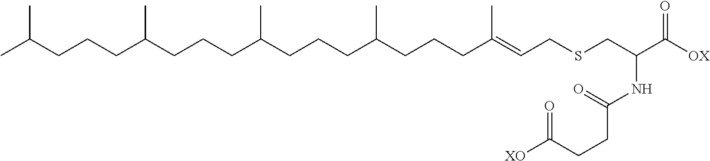
and any variations thereof, including all isomers, enantiomers and potential conversions to salts, esters, polymorphs, hydrates and/or solvates of the N-[1-Carboxy-2-((3,7,11,15,19-pentamethyl-eicos-2-enyl)sulfanyl)-ethyl]succinamic acid or salt.

15. The topical composition of claim 1, wherein the isoprenylcysteine is N-[1-Carboxy-2-((3,7,11,15-tetramethyl-hexadec-2-enyl)sulfanyl)-ethyl]succinamic acid or salt of formula: 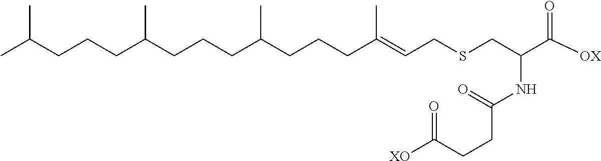
and any variations thereof, including all isomers, enantiomers and potential conversions to salts, esters, polymorphs, hydrates and/or solvates of the N-[1-Carboxy-2-((3,7,11,15-tetramethyl-hexadec-2-enyl)sulfanyl)-ethyl]succinamic acid or salt.

16. The topical composition of claim 1, wherein the isoprenylcysteine is N-[1-Carboxy-2-((3,7,11-trimethyl-dodec-2-enyl)sulfanyl)-ethyl]succinamic acid or salt of formula: 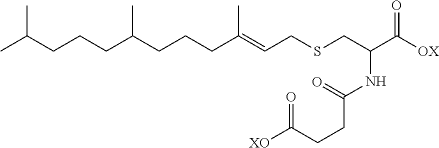
and any variations thereof, including all isomers, enantiomers and potential conversions to salts, esters, polymorphs, hydrates and/or solvates of the N-[1-Carboxy-2-((3,7,11-trimethyl-dodec-2-enyl)sulfanyl)-ethyl]succinamic acid or salt.

17. The topical composition of claim 1, wherein the isoprenylcysteine is Disodium Trimethyldodecenyl Succinoyl Cysteine, Disodium Tetramethylhexadecenyl Succinoyl Cysteine and/or Disodium Pentamethyleicosenyl Succinoyl Cysteine.

18. The topical composition of claim 1, wherein the isoprenylcysteine has a concentration from about 0.1 w/v % to about 10 w/v %.

19. The topical composition of claim 1, wherein the isoprenylcysteine has a concentration of about 3 w/v %.

20. A method for treating an area of skin to reduce DNA fragmentation induced by ultra-violet radiation comprising:
(a) topically applying a composition to the area of skin, the composition comprising:
(i) an extract of bidens pilosa;
(ii) isoprenylcysteine; and
(iii) a dermatologically acceptable carrier.

21. The method of claim 20, wherein the composition further comprises centella asiatica leaf cell extract.

22. The method of claim 20, wherein the composition further comprises one or more of aloe barbadensis leaf juice, natural albumen, octyldodecyl olivate, hydrolyzed quinoa, and tocopherol.

23. The method of claim 22, wherein the composition further comprises one or more of oryza sativa bran oil, Montanov™ L, Montanov™ 68, stearic acid, glycerin, glyceryl stearate, ricinus communis seed oil, cetyl alcohol, olus oil, chondrus crispus powder, sodium isostearoyl lactylate, sodium salt of pyroglutamic acid, polysorbate-20, Dermsoft™ LP, natural cucumber, and esseachem.

24. The method of claim 20, wherein the composition further comprises one or more jojoba esters, camellia sinensis leaf extract, hydrogenated lecithin, ubiquinone, bisabolol, terapeptide-30, and palmitoyl dipeptide-5 diaminobutyroyl hydroxythreonine.

25. The method of claim 23, wherein the composition further comprises aloe vera juice.

26. The method of claim 20, wherein the composition further comprises mauritia flexuosa fruit oil.

27. The method of claim 20 further comprising cleansing the area of the skin prior to applying the composition.

28. The method of claim 20 further comprising applying the composition once daily in the morning.

29. The method of claim 20 further comprising applying the composition once daily in the evening.

30. The method of claim 20 further comprising applying the composition once in the morning and once in the evening.

31. The method of claim 20 further comprising applying the composition as needed for moisturizing the area of skin.

32. The method of claim 20 further comprising applying the composition for treating the area of skin to reduce appearance of fine lines and wrinkles, discoloration, uneven texture, enlarged pores, aging or loose skin.

33. The method of claim 20, wherein the isoprenylcysteine has a concentration from about 0.1 w/v % to about 10 w/v %.

34. A topical composition comprising:
(a) extract of bidens pilosa;
(b) mauritia flexuosa fruit oil;
(c) jojoba esters;
(d) isoprenylcysteine;
(e) centella asiatica leaf cell extract; and
(f) tetrahexyldecyl ascorbate.

35. A topical composition comprising:
(a) extract of bidens pilosa;
(b) mauritia flexuosa fruit oil;
(c) centella asiatica leaf cell extract;
(d) isoprenylcysteine; and
(e) pancratium maritimum extract.

36. The topical composition of claim 34, wherein the isoprenylcysteine has a concentration from about 0.1 w/v % to about 10 w/v %.

37. The topical composition of claim 35, wherein the isoprenylcysteine has a concentration from about 0.1 w/v % to about 10 w/v %.
